(19) United States
(12) Patent Application Publication
(10) Pub. No.: US 2010/0297613 A1
(43) Pub. Date: Nov. 25, 2010

(54) Nucleic acid, amino acid encoded by said nucleic acid, probe comprising said nucleic acid or said amino acid, and screening method using said probe

(75) Inventors: Masahiro Ishiura, Nagoya-shi (JP); Kiyoshi Onai, Nagoya-shi (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA UNIVERSITY, Nagoya-shi, Aichi (JP)

(21) Appl. No.: 11/921,786

(22) PCT Filed: Jun. 5, 2006
(86) PCT No.: PCT/JP2006/311677

§ 371 Date: Dec. 7, 2007

(30) Foreign Application Priority Data
Jun. 9, 2005 (JP) 2005-169795

(51) Int. Cl. C12Q 1/68; C07H 21/04; C07K 14/415; C12N 15/63; C12P 21/02; C12N 5/10; C12N 1/19; C12N 1/21

(52) U.S. Cl. 435/6; 536/23.6; 536/24.3; 530/370; 435/320.1; 435/69.1; 435/419; 435/348; 435/325; 435/254.2; 435/252.3

(57) ABSTRACT

A nucleic acid involved in the control of the biological clock comprising following (a) or (b):
(a) a nucleic acid comprising a base sequence represented by base sequence numbers 1-2846 shown in SEQ ID No. 1 of the sequence listing, or
(b) a nucleic acid wherein a part of said base sequence represented by base sequence numbers 1-2846 is deleted, substituted or added, and having 80% homology with said base sequence.

### BACKGROUND OF THE INVENTION

1. Field of the Invention

The present invention relates to a nucleic acid, an amino acid encoded by said nucleic acid, a probe comprising said nucleic acid or said amino acid, and screening method using said probe, particularly a nucleic acid involved in the biological clock, an amino acid encoded by said nucleic acid, a probe comprising said nucleic acid or said amino acid, and a screening method using said probe.

2. Related Art

In order to adjust to daily fluctuation of light or temperature change associated with the day-night rotation of the environment, almost all organisms from cyanobacteria to humans have a biological clock (also known as the circadian clock or body clock), and biological activity of the organisms shows 24-hour-period rhythms (circadian rhythms). It has been revealed that the oscillation of these circadian rhythms is generated by both positive and negative feedback control of expression of the clock genes in cyanobacteria, Neurospora, Drosophila, mice, humans and the like (Dunlap, Cell, 96:271-290 (1999); Ishiura et al., Science 281: 1519-1523 (1998); Stanewsky, J. Neurosci. 54: 111-147 (2003)). There have been no similarities in the clock genes found among biological kingdoms, but the control mechanism of the rhythm oscillation is very similar. In plants, almost all physiological phenomena such as opening and closing of the stomata, nyctinasty of the leaves, hypocotyl elongation, photosynthesis, photoperiodic flowering, morphosis, various metabolic activities and the like are controlled by the biological clock (Lumsden and Millar, Biological Rhythms and Photoperiodism in Plants, Oxford: Bios Scientific Publisher (1998); McClung, Annu. Rev. Plant Physiol. Plant Mol. Biol. 52: 139-162 (2001)). Also, the biological clock controls gene expression as well. For example, it is known that in Arabidopsis, a model higher plant, the expression of about 6% of total genes follows circadian rhythms under constant light (Harmer et al., Science 290: 2110-2113 (2000)). However, the clock genes of plants have not been determined yet, and the molecular mechanism of the biological clock is virtually unknown.

In general, the requirements that the clock gene of a plant must satisfy are the following five points: (1) all circadian rhythms are lost due to the loss of function of one gene, (2) photoperiodism is lost due to the loss of function of one gene, (3) gene expression shows circadian rhythms under constant light and constant dark conditions, (4) all circadian rhythms are disrupted by the overexpression of the gene, (5) the gene controls the expression of itself through feedback control. However, no plant gene that satisfy all of these requirements have been found.

Regarding Arabidopsis, there are the theories that the CCA1 gene (Wang and Tobin, Cell 93: 1207-1217 (1998)), LHY gene (Schaffer et al., Cell 93: 1219-1229 (1998)), TOC1/APRR1 gene (Makino et al., Plant Cell Physiol. 43:58-69 (2000); Strayer et al., Science 289: 768-771 (2000)), and the ELF4 gene (Doyle et al., Nature 419: 74-77 (2002)) are clock genes (Young and Kay, Nat. Rev. Genet. 2: 702-715 (2001); Yanovsky and Kay, Nat. Rev. Mol. Cell Biol. 4: 265-275 (2003)). Also, in Arabidopsis, the genes have been found that influence parameters of the circadian rhythms (period, phase and amplitude of the rhythms), and they are the genes in relation to the photoreceptor and phototransduction system (Hayama and Coupland, Curr. Opin. Plant Biol. 6: 13-19 (2003); Yanovsky and Kay, Nat. Rev. Mol. Cell Biol. 4: 265-275 (2003)).

### SUMMARY OF THE INVENTION

However, these genes do not satisfy the requirements necessary for clock genes given above. The largest problem when regarding these genes as clock genes is that the loss of function of one gene does not cause the loss of circadian rhythms in all of them, and thereby these genes are not essential for rhythm oscillation. According to the situations as described above, it can be believed that the clock genes of plants have not been determined.

Therefore, the object of this invention is to provide genes that construct the biological clock capable of controlling physiological phenomena and biological activity, as well as proteins that are encoded by said genes.

In order to accomplish the above object, the inventors of this invention first performed a comprehensive and large-scale screening of clock mutants and obtained arrhythmic mutants pcl1-1 mutant and pcl1-2 mutant, and then found the nucleic acids and the proteins encoded by said nucleic acids according to this invention as the genes responsible for these mutants.

A nucleic acid involved in the control of the biological clock according to this invention is characterized by comprising following (a) or (b):
(a) a nucleic acid comprising a base sequence represented by base sequence numbers 1-2846 shown in SEQ ID No. 1 of the sequence listing, or
(b) a nucleic acid wherein a part of said base sequence represented by the base sequence numbers 1-2846 is deleted, substituted or added, and having 80% homology with said base sequence.

Furthermore, a nucleic acid involved in the control of the biological clock according to this invention is characterized by comprising following (a) or (b):
(a) a nucleic acid comprising a base sequence represented by base sequence numbers 1-4554 shown in SEQ ID No. 2 of the sequence listing, or
(b) a nucleic acid wherein a part of said base sequence represented by the base sequence numbers 1-4554 is deleted, substituted or added, and having 80% homology with said base sequence.

Furthermore, a nucleic acid involved in the control of the biological clock according to this invention is characterized by comprising following (a) or (b):
(a) a nucleic acid comprising a base sequence represented by base sequence numbers 1-4700 shown in SEQ ID No. 3 of the sequence listing, or
(b) a nucleic acid wherein a part of said base sequence represented by the base sequence numbers 1-4700 is deleted, substituted or added, and having 80% homology with said base sequence.

Furthermore, a nucleic acid involved in the control of the biological clock according to this invention is characterized by comprising following (a) or (b):
(a) a nucleic acid comprising a base sequence represented by base sequence numbers 1-1505 shown in SEQ ID No. 4 of the sequence listing, or
(b) a nucleic acid wherein a part of said base sequence represented by the base sequence numbers 1-1505 is deleted, substituted or added, and having 80% homology with said base sequence.

Furthermore, a nucleic acid involved in the control of the biological clock according to this invention is characterized by comprising following (a) or (b):
(a) a nucleic acid comprising a base sequence represented by base sequence numbers 1-400 shown in SEQ ID No. 5 of the sequence listing, or
(b) a nucleic acid wherein a part of said base sequence represented by the base sequence numbers 1-400 is deleted, substituted or added, and having 80% homology with said base sequence.

Furthermore, a nucleic acid involved in the control of the biological clock according to this invention is characterized by comprising following (a) or (b):
(a) a nucleic acid comprising a base sequence represented by base sequence numbers 1-641 shown in SEQ ID No. 6 of the sequence listing, or
(b) a nucleic acid wherein a part of said base sequence represented by base sequence numbers 1-641 is deleted, substituted or added, and having 80% homology with said base sequence.

Furthermore, a nucleic acid involved in the control of the biological clock according to this invention is characterized by comprising following (a) or (b):
(a) a nucleic acid comprising a base sequence represented by base sequence numbers 1-1400 shown in SEQ ID No. 7 of the sequence listing, or
(b) a nucleic acid wherein a part of said base sequence represented by base sequence numbers 1-1400 is deleted, substituted or added, and having 80% homology with said base sequence.

Furthermore, a probe according to this invention is characterized by comprising the nucleic acid described in any one of Claims 1-7.

In a preferred embodiment of the prove according to this invention, the probe is characterized by used for screening a gene involved in the control of the biological clock in living organisms.

Furthermore, a peptide fragment involved in the control of the biological clock, according to this invention is characterized by comprising following (a) or (b):
(a) a peptide fragment comprising an amino acid sequence represented by amino acid sequence numbers 1-323 shown in SEQ ID No. 8 of the sequence listing, or
(b) a peptide fragment wherein a part of said amino acid sequence shown in the SEQ ID No. 8 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-298 shown in SEQ ID No. 9 of the sequence listing, or
(b) a peptide fragment wherein a part of said amino acid sequence shown in the SEQ ID No. 9 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-238 shown in SEQ ID No. 10 of the sequence listing, or
(b) a peptide fragment wherein a part of said amino acid sequence shown in the SEQ ID No. 10 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-312 shown in SEQ ID No. 11 of the sequence listing, or
(b) a peptide fragment wherein a part of said amino acid sequence shown in the SEQ ID No. 11 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-70 shown in SEQ ID No. 12 of the sequence listing, or
(b) a peptide fragment wherein a part of said amino acid sequence in the SEQ ID No. 12 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-185 shown in SEQ ID No. 13 of the sequence listing, or
(b) a peptide fragment wherein a part of said amino acid sequence shown in the SEQ ID No. 13 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-314 shown in SEQ ID No. 14 of the sequence listing, or
(b) a peptide fragment wherein a part of said amino acid sequence shown in the SEQ ID No. 14 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-121 shown in SEQ ID No. 15 in the sequence listing, or
(b) a peptide fragment wherein a part of said amino acid sequence shown in the SEQ ID No. 15 is deleted, substituted or added, and having 80% homology with said amino acid sequence.

Furthermore, a peptide fragment involved in the control of the biological clock, according to this invention is characterized by comprising following (a) or (b):
(a) a peptide fragment comprising an amino acid sequence represented by amino acid sequence numbers 1-200 shown in SEQ ID No. 16 of the sequence listing, or
(b) a peptide fragment wherein a part of said amino acid sequence shown in the SEQ ID No. 16 is deleted, substituted or added, and having 80% homology with said amino acid sequence.

Furthermore, a probe according to this invention is characterized by comprising the peptide fragment described in any one of Claims 10-18.

Furthermore, a method for screening a gene involved in the control of the biological clock according to this invention is characterized by using the probe described in any one of Claims 8, 9 and 19.

In a preferred embodiment of the method for screening a gene involved in the control of the biological clock according to this invention, the screening is characterized by conducted using at least one selected from the group comprising in situ hybridization method, Southern hybridization method and determination of total base sequence.

In a preferred embodiment of the peptide fragment according to this invention, the peptide fragment is characterized by controlling the transcription of specific genes in the cell nucleus, and the oscillation and the stabilization of circadian rhythms.

In the other preferred embodiment of the peptide fragment according to this invention, the peptide fragment is characterized by having a DNA-binding motif belonging to the GARP family.

Furthermore, a composition for control of the biological clock according to this invention is characterized by comprising the peptide fragment described in any one of Claims 10-18.

Furthermore, a vector according to this invention is characterized by comprising DNA or RNA described in any one of Claims 1-7.

Furthermore, a transformant according to this invention characterized by holding the DNA or the RNA described in any one of Claims 1-7 in a manner that enables its expression.

Furthermore, a method for producing a peptide according to this invention is characterized by comprising a step of culturing the transformant according to this invention.

Furthermore, a peptide fragment involved in the control of the biological clock according to this invention is characterized by comprising following (a) or (b):
(a) a peptide fragment comprising an amino acid sequence represented by amino acid sequence numbers 1-210 shown in SEQ ID No. 8 of the sequence listing, or
(b) a peptide fragment wherein a part of said amino acid sequence shown in the SEQ ID No. 8 is deleted, substituted or added, and having 80% homology with said amino acid sequence.

Furthermore, a peptide fragment involved in the control of the biological clock according to this invention is characterized by comprising following (a) or (b):
(a) a peptide fragment comprising an amino acid sequence represented by amino acid sequence numbers 1-143 shown in SEQ ID No. 8 of the sequence listing, or
(b) a peptide fragment wherein a part of said amino acid sequence shown in the SEQ ID No. 8 is deleted, substituted or added, and having 80% homology with said amino acid sequence.

Furthermore, a probe according to this invention is characterized by comprising the peptide fragment described in Claim 28 or 29.

Furthermore, a method for screening of a peptide fragment involved in the control of the biological clock according to this invention is characterized by using the probe described in Claim 30.

The present invention has an advantage that the same manipulation in rice as in Arabidopsis is enable since OsPCL1, the homologous gene of the PCL1 gene, could be isolated from the rice genome. Furthermore, the cDNA of the homologous gene of the PCL1 gene has been found in potato, tomato, tobacco, corn and pine. These nucleic acids and these peptide fragments according to this invention have an advantage that the artificial manipulation of the PCL1 gene, or PCL1-homologous genes or PCL1-similar genes according to this invention, which can be easily assumed that they function as the clock gene in each of the plants, can be provide the fundamental research materials for controlling various physiological phenomena and biological activity of the higher plant including photoperiodic flowering.

Furthermore, this invention has the other advantage of that development of the agrichemicals for controlling the activity of the clock protein paves the way for efficient control of the biological activity of plants in arbitrary time since the amino acid sequences of the proteins encoded by the clock genes have been revealed. Therefore, it is possible to proceed efficiently in the increase of productivity and the breed improvement of agricultural products.

### BRIEF DESCRIPTION OF THE DRAWINGS

FIG. 1 shows GI::LUC⁺ bioluminescence pattern of pcl1 mutant under constant light condition or constant dark condition. The GI::LUC⁺ bioluminescence of wild-type strain G-38 (blue), the pcl1-1 mutant (red) and the pcl1-2 mutant (brown) were measured under constant light condition (a) or constant dark condition (b). Hatched white bar and solid black bar in the figure respectively represents the light period and the dark period. Plots in the figure are the mean value±standard deviation of 96 individuals.

FIG. 2 shows GI::LUC⁺ bioluminescence pattern of pcl1 mutant under light/dark cycle condition or temperature cycle condition. The GI::LUC⁺ bioluminescence of wild-type strain G-38 (blue) and the pcl1-1 mutant (red) was measured using cycles of 12-hour light period/12-hour dark period light-dark (a; 12L12D) or cycles of 12-hour 22° C./12-hour 17° C. temperature (b; 22° C.17° C.). Hatched white bars and solid blacks bar in the figure respectively represent the light period and dark period. Yellow bars and light blue bars in the figure respectively represent the 22° C. period and the 17° C. period. Plots in the figure are the mean value±standard deviation of 96 individuals.

FIG. 3 shows nyctinasty of the leaves of pcl1 mutant. The nyctinasty of the leaves of wild-type strain G-38 (blue) and the pcl1-1 mutant (red) were measured under constant light condition. Hatched white bar in the figure represents the light period. In the plots, the position of cotyledon in the Y-axis direction at the start of the measurement was taken to be 0.

FIG. 4 shows Northern blot analysis of the expression of GI, CAB2, TOC1, ELF4, CCA1 and LHY genes in pcl1 mutant. Northern blot analysis of the GI mRNA (a), CAB2 mRNA (b), TOC1 mRNA (c), ELF4 mRNA (d), CCA1 mRNA (e) and LHY mRNA (f) levels in the cells of wild-type strain G-38 (blue) and the pcl1-1 mutant (red) were performed under constant light condition.

FIG. 5 shows photoperiodic flowering of pcl1 mutant. Wild-type Col-0 strain and G-38 strain, and the pcl1 mutants pcl1-1 and pcl1-2 were cultured under 16-hour light period/8-hour dark period (16L8D; light green) long daytime condition or 10-hour light period/14-hour dark period (10L14D; deep green) short daytime condition, thereafter the flowering timing was determined.

FIG. 6 shows map-based cloning of PCL1 gene and structure of the PCL1 gene.

FIG. 7 shows structure of PCL1 protein. The structure of the PCL1 protein and alignment of the amino acid residues of the GARP motifs of the PCL1 protein, ARR1 protein and ARR10 protein are shown. The amino acid residue matching the PCL1 protein is encircled in red.

FIG. 8 shows localization of PCL1 protein in the cells. (a, c, e) The fluorescence of GFP observed using the fluorescence microscope. (b, d, e) are images of the cells observed using the optical microscope. (a, b) The cases when the expression of only GFP was caused. (c, d) The cases when the expression of the PCL1-GFP1 fused protein is caused. (e, f) The cases when the expression of the GFP-PCL1 fused protein is caused.

FIG. 9 shows comparison of PCL1 protein with PCL1-similar proteins and PCL1-homologous proteins. Multiple alignment of each of the amino acid sequences of the PCL1 protein of Arabidopsis (Arabidopsis thaliana), PCL1-similar protein of Arabidopsis (Arabidopsis thaliana) PCLL, PCL1-homologous protein of rice (Oryza sativa) OsPCL1, PCL1-homologous protein of tobacco (Nicotiana benthamina) NbPCL1, PCL1-homologous protein of tomato (Lycopersicon esculentum) LePCL1, PCL1-homologous protein of potato (Solanum tuberosum) StPCL1, and PCL1-homologous protein of pine (Pinus taeda) PtPCL1, is shown. Asterisks (*) are added to matching amino acid residues, and dots (.) are added to similar amino acid residues. Gaps are represented by bars (-). Red lines are added on top of the amino acid residues to show GARP motifs. Amino acid residues written in red in the PCL1 amino acid sequence are amino acid residues that are changed to stop codons in the pcl1-1 mutation and pcl1-2 mutation. FIG. 10 shows Northern blot analysis of the expression of PCL1 gene. Northern blot analysis of the PCL1 mRNA level in the cells of wild-type strain G-38 (blue) and pcl1-1 mutant (red) under constant light condition was performed.

FIG. 11 shows PCL1::LUC⁺ bioluminescence rhythms. The PCL1::LUC⁺ bioluminescence of wild-type strain G-38 (blue) and the pcl1-1 mutant (red) were measured under constant light condition (a) or constant dark condition (b). Hatched white bar and solid black bar in the figure respectively represent the light period and dark period. Plots in the figure are the mean value±standard deviation of 48 individuals.

FIG. 12 shows GI::LUC⁺ bioluminescence pattern and nyctinasty of the leaves of PCL1-ox plants. (a, b) the GI::LUC⁺ bioluminescence of wild-type strain G-38 (blue) and the PCL1-ox plants (green) was measured under constant light condition (a) or constant dark condition (b). Hatched white bars and solid black bar in the figure respectively represent the light period and dark period. Plots in the figure are the mean value±standard deviation of 96 individuals (a) or 48 individuals (b), respectively. (c) The nyctinasty of the leaves of wild-type strain G-38 (blue) and the PCL1-ox plants (green) were measured under constant light condition. In the plots, the position of cotyledon in the Y-axis direction at the start of the measurement was taken to be 0. Hatched white bars in the figure represent the light period. Plots in the figure are the mean value±standard deviation of 12 individuals.

FIG. 13 shows Northern blot analysis of the expression of the endogenous PCL1 gene in PCL1-ox plants. Northern blot analysis of the levels of PCL1 mRNA derived from the endogenous PCL1 gene in the cells of wild-type strain G38 (blue), the pcl1-1 mutant (red), and the PCL-ox plants (green) under constant light condition was performed.

FIG. 14 shows model of the biological clock in plants. The gene control manners discovered in this invention are shown in red, and the known gene control manners are shown in blue. Arrows represent the promotion of gene expression, and horizontal lines represent the suppression of gene expression. The negative self-feedback loop of the expression of the PCL1 gene is essential for clock oscillation, and this is the central oscillator of the plant clock.

FIG. 15-1 shows the sequences of PCL1 gene and PCL1 protein (Arabidopsis thaliana).

FIG. 15-2 shows the sequences of PCL1 gene and PCL1 protein (Arabidopsis thaliana).

FIG. 15-3 shows the sequences of PCL1 gene and PCL1 protein (Arabidopsis thaliana).

FIG. 15-4 shows the sequences of PCL1 gene and PCL1 protein (Arabidopsis thaliana).

FIG. 16-1 shows the sequences of PCLL gene and PCLL protein (Arabidopsis thaliana).

FIG. 16-2 shows the sequences of PCLL gene and PCLL protein (Arabidopsis thaliana).

FIG. 16-3 shows the sequences of PCLL gene and PCLL protein (Arabidopsis thaliana).

FIG. 16-4 shows the sequences of PCLL gene and PCLL protein (Arabidopsis thaliana).

FIG. 17-1 shows the sequences of OsPCL1 gene and OsPCL1 protein (Oryza sativa).

FIG. 17-2 shows the sequences of OsPCL1 gene and OsPCL1 protein (Oryza sativa).

FIG. 17-3 shows the sequences of OsPCL1 gene and OsPCL1 protein (Oryza sativa).

FIG. 17-4 shows the sequences of OsPCL1 gene and OsPCL1 protein (Oryza sativa).

FIG. 18-1 shows the sequences of NbPCL1 gene and NbPCL1 protein (Nicotiana benthamina).

FIG. 18-2 shows the sequences of NbPCL1 gene and NbPCL1 protein (Nicotiana benthamina).

FIG. 18-3 shows the sequences of NbPCL1 gene and NbPCL1 protein (Nicotiana benthamina).

FIG. 19 shows the sequences of NtPCL1 gene and NtPCL1 protein (Nicotiana tabacum).

FIG. 20-1 shows the sequences of LePCL1 gene and LePCL1 protein (Lycopersicon esculentum).

FIG. 20-2 shows the sequences of LePCL1 gene and LePCL1 protein (Lycopersicon esculentum).

FIG. 21-1 shows the sequences of StPCL1 gene and StPCL1 protein (Solanum tuberosum).

FIG. 21-2 shows the sequences of StPCL1 gene and StPCL1 protein (Solanum tuberosum).

FIG. 21-3 shows the sequences of StPCL1 gene and StPCL1 protein (Solanum tuberosum).

FIG. 22 shows PCLL::LUC⁺ bioluminescence rhythms. The bioluminescence of the wild-type PCLL::LUC⁺ luminescent reporter strain was measured under constant light (a) or constant dark (b). Hatched white bar and solid black bar in the figure respectively represents the light period and dark period. Plots in the figure are the mean value±standard deviation of 96 individuals.

FIG. 23 shows GI::LUC⁺ bioluminescence pattern of the PCLL-ox plants. The GI::LUC⁺ bioluminescence of the PCL-ox plants was measured under constant light. Hatched white bar in the figure represents the light period. Plots in the figure are the mean value±standard deviation of 96 individuals.

### BEST MODE FOR CARRYING OUT THE INVENTION

First, the nucleic acid according to this invention will be described below. The nucleic acid according to this invention involved in the control of the biological clock comprises following (a) or (b):
(a) a nucleic acid comprising a base sequence represented by base sequence numbers 1-2846 shown in SEQ ID No. 1 of the sequence listing, or
(b) a nucleic acid wherein a part of said base sequence of the base sequence numbers 1-2846 is deleted, substituted or added, and having 80%, preferably 90% or more preferably 95% homology with said base sequence. Said nucleic acid as mentioned above is derived from Arabidopsis. The nucleic acid according to this invention also includes a nucleic acid wherein a part of said base sequence is deleted, substituted or added, and having 80%, preferably 90% or more preferably 95% homology with said base sequence. It means that even such a nucleic acid wherein a part of said base sequence is deleted, substituted or added can be used, for example, as a probe for searching a gene involved in the control of the biological clock, as described below. Moreover, in this description, the term “a gene wherein a part of the base sequence is deleted, substituted or added” means a gene having a sequence wherein 10 or less, preferably 7 or less, or more preferably 3 or less bases are deleted, substituted or added in the base sequence. Furthermore, such a gene forms hybrids with the gene shown in SEQ ID No. 1 of the sequence listing under stringent conditions. Even such a gene is also included in the gene according to this invention as long as it is a factor involved in the control of the biological clock.

Furthermore, a nucleic acid according to this invention involved in the control of the biological clock comprises following (a) or (b):
(a) a nucleic acid comprising a base sequence represented by base sequence numbers 1-4554 shown in SEQ ID No. 2 of the sequence listing, or
(b) a nucleic acid wherein a part of said base sequence of the base sequence numbers 1-4554 is deleted, substituted or added, and having 80%, preferably 90%, or more preferably 95% homology with said base sequence. Said nucleic acid as mentioned above is derived from Arabidopsis. The nucleic acid according to this invention also includes a nucleic acid wherein a part of said base sequence is deleted, substituted or added, and having 80%, preferably 90%, or more preferably 95% homology with said base sequence. It means that even such a nucleic acid wherein a part of said base sequence is deleted, substituted or added can be used, for example, as a probe for searching a gene involved in the control of the biological clock. Furthermore, said gene forms hybrids with the gene shown in SEQ ID No. 2 of the sequence listing under stringent conditions. Such a gene is also included in the gene according to this invention as long as it is a factor involved in the control of the biological clock.

Furthermore, a nucleic acid according to this invention involved in the control of the biological clock comprises following (a) or (b):
(a) a nucleic acid comprising a base sequence represented by base sequence numbers 1-4700 shown in SEQ ID No. 3 of the sequence listing,
(b) a nucleic acid wherein a part of said base sequence of the base sequence numbers 1-4700 is deleted, substituted or added, and having 80%, preferably 90% or more preferably 95% homology with said base sequence. Said nucleic acid as mentioned above is derived from the rice genome. The term “a gene wherein a part of the gene is deleted, substituted or added means a gene having a sequence wherein 10 or less, preferably 7 or less, or more preferably 3 or less bases are deleted, substituted or added in the base sequence shown in SEQ ID No. 3. Such a gene forms hybrids with the gene shown in SEQ ID No. 3 of the sequence listing.

Furthermore, a nucleic acid according to this invention involved in the control of the biological clock comprises following (a) or (b):
(a) a nucleic acid comprising a base sequence represented by base sequence numbers 1-1505 shown in SEQ ID No. 4 of the sequence listing,
(b) a nucleic acid wherein a part of said base sequence of the base sequence numbers 1-1505 is deleted, substituted or added, and having 80% homology with said base sequence. Said nucleic acid as mentioned above is derived from tobacco. The nucleic acid according to this invention also includes a nucleic acid wherein a part of said base sequence is deleted, substituted or added, and having 80%, preferably 90% or more preferably 95% homology with said base sequence. It means that even such a nucleic acid wherein a part of said base sequence is deleted, substituted or added can be used, for example, as a probe for searching a gene involved in the control of the biological clock. Furthermore, such a gene forms hybrids with the gene shown in SEQ ID No. 4 of the sequence listing under stringent conditions. Such a gene is also included in the gene according to this invention as long as it is a factor involved in the control of the biological clock.

Furthermore, a nucleic acid according to this invention involved in the control of the biological clock comprises following (a) or (b):
(a) a nucleic acid comprising a base sequence represented by base sequence numbers 1-400 shown in SEQ ID No. 5 of the sequence listing, or
(b) a nucleic acid wherein a part of said base sequence of the base sequence numbers 1-400 is deleted, substituted or added, and having 80% homology with said base sequence. Said nucleic acid as mentioned above is derived from another strain of tobacco. The nucleic acid according to this invention also includes a nucleic acid wherein a part of said base sequence is deleted, substituted or added, and having 80%, preferably 90% or more preferably 95% homology with said base sequence. It means that even such a nucleic acid wherein a part of said base sequence is deleted, substituted or added can be used, for example, as a probe for searching a gene involved in the control of the biological clock. Furthermore, said gene forms hybrids with the gene shown in SEQ ID No. 5 of the sequence listing under stringent conditions. Such a gene is also included in the gene according to this invention as long as it is a factor involved in the control of the biological clock.

Furthermore, a nucleic acid according to this invention involved in the control of the biological clock comprises following (a) or (b):
(a) a nucleic acid comprising a base sequence represented by base sequence numbers 1-641 shown in SEQ ID No. 6 of the sequence listing,
(b) a nucleic acid wherein a part of said base sequence of the base sequence numbers 1-641 is deleted, substituted or added, and having 80% homology with said sequence. Said nucleic acid as mentioned above is derived from tomato. The nucleic acid according to this invention also includes a nucleic acid wherein a part of said base sequence is deleted, substituted or added, and having 80%, preferably 90% or more preferably 95% homology with said base sequence. It means that even such a nucleic acid wherein a part of said base sequence is deleted, substituted or added can be used, for example, as a probe for searching a gene involved in the control of the biological clock. Furthermore, said gene forms hybrids with the gene shown in SEQ ID No. 6 of the sequence listing under stringent conditions. Such a gene is also included in the gene according to this invention as long as it is a factor involved in the control of the biological clock.

Furthermore, a nucleic acid according to this invention involved in the control of the biological clock comprises following (a) or (b):
(a) a nucleic acid comprising a base sequence represented by base sequence numbers 1-1400 shown in SEQ ID No. 7 of the sequence listing, or
(b) a nucleic acid wherein a part of said base sequence of the base sequence numbers 1-1400 is deleted, substituted or added, and having 80% homology with said base sequence. Said nucleic acid as mentioned above is derived from potato. The nucleic acid according to this invention also includes a nucleic acid wherein a part of said base sequence is deleted, substituted or added, and having 80%, preferably 90% or more preferably 95% homology with said base sequence. It means that even such a nucleic acid wherein a part of said base sequence is deleted, substituted or added can be used, for example, as a probe for searching a gene involved in the control of the biological clock. Furthermore, said gene forms hybrids with the gene shown in SEQ ID No. 7 of the sequence listing under stringent conditions. Such a gene is also included in the gene according to this invention as long as it is a factor involved in the control of the biological clock.

Next, a peptide fragment according to this invention involved in the control of the biological clock will be described below. The peptide fragment according to this invention involved in the control of the biological clock comprises following (a) or (b):
(a) a peptide fragment comprising an amino acid sequence represented by amino acid sequence numbers 1-323 shown in SEQ ID No. 8 of the sequence listing, or
(b) a peptide fragment wherein a part of said amino acid sequence shown in the SEQ ID No. 8 is deleted, substituted or added, and having 80% homology with said amino acid sequence. Said peptide fragment as mentioned above is derived from Arabidopsis. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, in this description, the term “an amino acid wherein a part of the amino acid sequence is deleted, substituted or added” means an amino acid sequence having a sequence wherein 10 or less, preferably 7 or less, or more preferably 3 or less amino acids are deleted, substituted or added in the amino acid sequence. Even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-298 shown in SEQ ID No. 9 of the sequence listing, or
(b) a peptide fragment wherein a part of said amino acid sequence shown in the SEQ ID No. 9 is deleted, substituted or added, and having 80% homology with said amino acid sequence. Said peptide fragment as mentioned above is derived from Arabidopsis. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-238 shown in SEQ ID No. 10 of the sequence listing, or
(b) a peptide fragment wherein a part of said amino acid sequence shown in the SEQ ID No. 10 is deleted, substituted or added, and having 80% homology with said amino acid sequence. Said peptide fragment as mentioned above is derived from rice. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-312 shown in SEQ ID No. 11 of the sequence listing, or
(b) a peptide fragment wherein a part of said amino acid sequence shown in the SEQ ID No. 11 is deleted, substituted or added, and having 80% homology with said amino acid sequence. Said peptide fragment as mentioned above is derived from tobacco. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-70 shown in SEQ ID No. 12 of the sequence listing, or
(b) a peptide fragment wherein a part of said amino acid sequence shown in the SEQ ID No. 12 is deleted, substituted or added, and having 80% homology with said amino acid sequence. Said peptide fragment as mentioned above is derived from another strain of tobacco. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-185 shown in SEQ ID No. 13 of the sequence listing, or
(b) a peptide fragment wherein a part of said amino acid sequence shown in the SEQ ID No. 13 is deleted, substituted or added, and having 80% homology with said amino acid sequence. Said peptide fragment as mentioned above is derived from tomato. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-314 shown in SEQ ID No. 14 of the sequence listing, or
(b) a peptide fragment wherein a part of said amino acid sequence shown in the SEQ ID No. 14 is deleted, substituted or added, and having 80% homology with said amino acid sequence. Said peptide fragment as mentioned above is derived from potato. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-121 shown in SEQ ID No. 15 of the sequence listing, or
(b) a peptide fragment wherein a part of said amino acid sequence shown in the SEQ ID No. 9 is deleted, substituted or added, and having 80% homology with said amino acid sequence. Said peptide fragment as mentioned above is derived from pine. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-200 shown in SEQ ID No. 16 of the sequence listing, or
(b) a peptide fragment wherein a part of said amino acid sequence shown in the SEQ ID No. 16 is deleted, substituted or added, and having 80% homology with said amino acid sequence. Said peptide fragment as mentioned above is derived from corn. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-210 shown in SEQ ID No. 8 of the sequence listing, or
(b) a peptide fragment wherein a part of said amino acid sequence represented by amino acid sequence numbers 1-210 shown in the SEQ ID No. 8 is deleted, substituted or added, and having 80% homology with said amino acid sequence. Said peptide fragment as mentioned above is derived from Arabidopsis. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction.

Furthermore, a peptide fragment according to this invention involved in the control of the biological clock comprises following (a) or (b):
(a) a peptide fragment comprising an amino acid sequence represented by amino acid sequence numbers 1-143 shown in SEQ ID No. 8 of the sequence listing, or
(b) a peptide fragment wherein a part of said amino acid sequence represented by amino acid sequence numbers 1-143 shown in the SEQ ID No. 8 is deleted, substituted or added, and having 80% homology with said amino acid sequence. Said peptide fragment as mentioned above is derived from Arabidopsis. The peptide fragment according to this invention also includes the peptide fragment wherein a part of said amino acid sequence is deleted, substituted or added, and having 80%, preferably 90% or more preferably 95% homology with said amino acid sequence. It means that even such a peptide fragment wherein a part of said amino acid sequence is deleted, substituted or added, for example, can be used as a probe for searching a peptide fragment involved in the control of the biological clock. Moreover, even such an amino acid sequence can also be used for the immunoassay using antigen-antibody reaction. These sequences have, as can be understood from comparing the peptide fragments according to this invention, very high homology, and it can be derived that these regions are very important for the function of the biological clock. Therefore, these important regions can be, for example using them as probes, utilized in further elucidation of the biological clock.

The method for purification and isolation of the above-mentioned nucleic acids and peptide fragments according to this invention will be described below. The methods for purification and isolation of the above-mentioned nucleic acids and peptide fragments are not particularly limited, the nucleic acids and the peptide fragments can be purified and isolated according to the following procedure.

In order to measure automatically the bioluminescence rhythms of multiple samples of the higher plant and analyze the measured data in one measurement, a bioluminescence real-time monitoring/screening system comprising two types of bioluminescence measurement device (Okamoto et. al., Anal. Biochem. 340: 187-192 (2005); Okamoto et al., Plant Cell Environ. 28: 1305-1315 (2005); Japanese published unexamined patent No. 2004-267058; Japanese published unexamined patent No. 2005-143371) and a rhythm analysis program (Okamoto et al., Anal. Biochem. 340: 193-200 (2005); Japanese Patent number 3787631) can be used.

Using these, for example, bioluminescence reporter gene cassette (GI::LUC⁺) connecting the promoter region of the GI gene (Fowler et al., EMBO J. 18: 4679-4688 (1999); Park et al., Science 285: 1579-1582 (1999)) of Arabidopsis, which is a clock-related gene of Arabidopsis and is known to show circadian rhythms in gene expression, to the coding region of the modified version of the firefly luciferase gene (LUC⁺; Promega Corp.) (LUC⁺ (registered trademark), Promega (registered trademark)) was constructed, then this gene cassette was introduced into the wild-type Arabidopsis genome, thereby bioluminescence reporter strain (G-38 strain) was constructed. The seeds of the constructed bioluminescence reporter strain G-38 were mutagen treated using ethyl methanesulfonate (EMS), the rhythm mutants were screened by measuring the bioluminescence rhythms of 50,000 plants of the second generation (M₂ plants) after the mutagen treatment, and five arrhythmic mutants having completely lost the bioluminescence rhythms were isolated. In these arrhythmic mutants, under both constant light condition and constant dark condition, bioluminescence rhythms of the GI::LUC⁺ luminescence reporter gene are arrhythmic, the nyctinasty of the leaves was also arrhythmic, each of these arrhythmic mutations were recessive single-gene mutations, and could be classified into three complementary groups PHYTOCLOCK 1 (PCL1), PCL2, and PCL3. The PCL1, PCL2, and PCL3 genes were respectively believed to encode the clock gene of the plants. The PCL1 gene, one of the PCL genes, was identified using the map-based cloning method in the following procedure. The F₃ homozygots (ecotype Col-0) and wild-type Ler strain were crossbred to obtain the second-generation (F₂) seeds. The F₂ plants were cultured, then the bioluminescence of the GI::LUC⁺ reporter gene the plants was measured under constant light condition, and homozygots carrying pcl1-1 in homo were selected. Then, using the polymorphic markers (CAPS marker and SSLP marker) between the Col-0 and Ler published at TAIR website (http://www.arabidopsis.org/) and SNP polymorphic marker released from Monsanto Arabidopsis Polymorphism Collection, the recombination rates between the pcl1-1 mutant and the polymorphic markers were calculated. Polymorphic markers having lower recombination rates are closer to the PCL1 gene physically, so the position of the PCL1 gene on the chromosomes was determined by examining the recombination rate for various markers. As a result, the physical position of the PCL1 gene was determined to be at the area of about 150 kb between the SNP marker F18L15-1 (containing SNP numbers CER468139 to CER 468143 of Monsanto Arabidopsis Polymorphism Collection) and the CAPS marker TOPP5 on the third chromosome. By determining and comparing the base sequences of wild-type Col-0 strain and G-38 strain, and arrhythmic mutants pcl1-1 and pcl1-2 in this area of about 150 kb, base substitution was found on the gene indexed at index number At3g46640 on the database at the TAIR website for both pcl1-1 and pcl1-2, so this was concluded to be the PCL1 gene. The structure of the PCL1 gene was determined by comparing the base sequence of the full-length cDNA and the genome DNA sequence published at RIKEN (RARGE; http://rarge.gsc.riken.go.jp/) and TAIR websites. The amino acid sequence of the proteins can easily be deduced from the base sequence of the genome DNA or the cDNA.

Moreover, according to common procedure, the base sequence of the nucleic acid can be determined by, for example, dye-terminator method. Alternatively, the base sequences of the genes that encode similar proteins or homolog proteins can be deduced from amino acid sequences of the proteins according to this invention, various oligonucleotides can be synthesized on the basis of the deduction, and these oligonucleotides can be used as PCR primers in the PCR method to amplify the genes from genome DNA or cDNA. Furthermore, the base sequences of the genes that encode similar proteins or homolog proteins can be deduced from the amino acid sequences of the proteins according to this invention, various oligonucleotides can be synthesized on the basis of the deduction, and these oligonucleotides can be used in various hybridization methods to identify the genes. Next, a probe according to this invention will be described below.

As a method for using the probe according to this invention, the genome DNA, genome DNA library, cDNA library, RNA and the like of the biological species wherefrom one wants to isolate the above-mentioned nucleic acids are amplified directly or by the PCR method and fixed on a polymer film by blotting, then the probe according to this invention may be hybridized with them. Alternatively, the cells of the biological species wherefrom one wants to isolate the above-mentioned nucleic acids may be fixed, and the probe according to this invention may be hybridized directly with the chromosomes in the cells. The method for hybridization is not particularly limited by conventional methods, however, Southern blotting method, in situ hybridization method, base sequence determination method, colony hybridization method, plaque hybridization method, Northern hybridization method, and the like can be listed, for example. In situ hybridization method is preferable from the viewpoint of its ability for fast and precise screening. The in situ hybridization method can include fluorescence in situ hybridization method (hereinafter referred to as FISH method), radioisotope in situ hybridization method, and the like. From the viewpoint of not requiring the RI facility, the FISH method is preferable. A general outline of the FISH method is as follows, for example: a chromosome sample is prepared on a slide glass, a labeled probe is hybridized with the sample, and then the sample is observed through direct microscopic examination.

Moreover, the support medium used for the hybridization of the probe according to this invention can include thin films, powders, particulates, gel, beads and fibers, as well as fluid dispersion, emulsion and the like. These may be used by filling a suitable column. Among these, thin films are preferable, and for example, nitrocellulose films or nylon films are preferable.

Here, examples of the labels used in the probe according to this invention will be described below. As examples of the labels, ones known to a person skilled in the art may be used, and the labels include, without limitation, radioactive atoms such as ³²P and ³⁵S, a biotin group, an adipic group, or enzymes, fluorescent labels and the like, as well as an antigen if an antigen-antibody system is used. These are also included in the scope of this invention.

Moreover, in the case of the nucleic acid probe, the base length of the probe varies depending on the screening method used, and is not to be limited particularly.

As for the above peptide fragments, in preferred embodiments, said peptide fragments control the transcription of certain genes in the nucleus of cells, and oscillation and stabilization of circadian rhythms. This comes from the fact that through the keen examination of the inventors of this invention, it has been revealed that the nucleic acid according to this invention is one of the genes involved in the control of the biological clock. The genes according to this invention serve as the core of the biological clock, and at the same time, they work as a transcription factor for other genes, and perform the oscillation and stabilization of circadian rhythms of living organisms.

Furthermore, in a preferred embodiment of the peptide fragments according to this invention, the above-mentioned peptide fragments have a DNA-binding motif belonging to the GARP family. It is believed that the circadian rhythms are formed by binding with other genes via said DNA-binding motif and functioning as a transcription factor.

Furthermore, a composition for controlling the biological clock according to this invention comprises the peptide fragment, a DNA fragment or a RNA fragment according to this invention and promotes or suppresses the biological clock function activity of the cell, and there are no limitations in the mode of the compositions for controlling the biological clock. The targets of the compositions for controlling the biological clock according to this invention are individual organisms or cultured cells, cell extracts, in vitro remodeling system of the biological clock of acellular systems, and the like.

Furthermore, a vector according to this invention comprises the above-mentioned nucleic acids (DNA or RNA) according to this invention. Furthermore, a transformant according to this invention hold the nucleic acid (DNA or RNA) according to this invention in a manner that enables its expression. Moreover, a method for producing a peptide according to this invention comprises a step of culturing the transformant according to this invention.

Hereinafter, the production of the recombinant vector according to this invention will be described.

The recombinant vector according to this invention can be obtained by connecting a gene involved in the biological clock according to this invention to a suitable vector. Any vector may be used as long as it is able to make the host produce the protein involved in the biological clock expressed by the gene involved in the biological clock according to this invention in the host to be transformed. For example, vectors such as plasmids, cosmids, phages, viruses, chromosomal integration types, artificial chromosomes and the like may be used. The above vectors may include marker genes that make it possible to select the transformed cells. The marker genes can include, for example, genes that complement auxotrophy of the host such as URA3 or niaD, genes that show resistance to drugs such as ampicillin, kanamycin, oligomycin, tetracycline, chloramphenicol, hygromycin B, Basta (registered trademark), and the like. Moreover, the recombinant vector preferably comprises a promoter or other control sequences (for example, enhancer sequence, terminator sequence, polyadenylation sequence and the like) that are able to express the gene according to this invention in the host cells. In particular, the promoter can include, for example, GAL1 promoter, amyB promoter, lac promoter, tac promoter, trc promoter, CaMV35S promoter and the like.

Furthermore, a transformant according to this invention are obtained by transforming the host with the recombinant vector according to this invention. The host is not limited particularly as long as it can produce the protein involved in the biological clock according to this invention. For example, the host may be plant-type cells such as cultured cells BY-2 of tobacco, cultured cells of Arabidopsis, tobacco plants or Arabidopsis plants, cultured cells of insect, animal cells, yeast such as fission yeast or budding yeast, filamentous fungi such as Aspergillus sojae Aspergillus oryzae, Aspergillus niger or Neurospora, or bacteria such as E. coli, Bacillus, or cyanobacteria. Transformation can be performed using methods known to those skilled in the art depending on the host used. In the case of plant-type cells, for example, a transformation method using Agrobacterium (Horsch et al., Science 227: 1229-1231 (1985); Hooykaas and Schilperoort, Plant Mol. Biol. 19: 15-38 (1992); Clough and Bent, Plant J. 16:735-743 (1998)) and the like can be used. In the case of yeast, for example, lithium acetate method (Ausubel et al., Current protocols in molecular biology. Greene Publishing Assoc and Wiley-Interscience, New York (1987); Methods Mol. Cell. Biol. 5: 255-269 (1995)) and the like can be used. In the case of filamentous fungi, for example, the method described in Mol. Gen. Genet. 218: 99-104 (1989) which uses polyethyleneglycol and calcium chloride after protoplastization can be used. In the case of using bacteria, for example, natural transformation method (Ausubel et al., Current protocols in molecular biology. Greene Publishing Assoc and Wiley-Interscience, New York (1987); Onai et al., Mol. Genet. Genomics 271: 50-59 (2004)), calcium chloride method (Ausubel et al., Current protocols in molecular biology. Greene Publishing Assoc and Wiley-Interscience, New York (1987)), electroporation (Ausubel et al., Current protocols in molecular biology. Greene Publishing Assoc and Wiley-Interscience, New York (1987); Methods Enzymol. 194: 182-187 (1990)) and the like may be used.

A method for producing the protein involved in the biological clock according to this invention comprises the steps of: culturing the transformant according to this invention; and collecting the protein involved in the biological clock from the obtained culture. A culture medium and a culture method are chosen to suit the type of host and the expression control sequence in the recombinant vector. For example, when the host is a plant-type cultured cell, and the expression control sequence is the CaMV35S promoter, the protein involved in the biological clock according to this invention can be produced, for example by culturing the cell in a medium that contains sucrose. Moreover, for example, when the host is plants, and the expression control sequence is the CaMV35S promoter, the protein involved in the biological clock according to this invention can be produced, for example, by culturing the plants in soil. Moreover, for example, when the host is yeast, and the expression control sequence is the GAL1 promoter, the protein involved in the biological clock according to this invention can be produced, for example, by preculturing the yeast cells in liquid minimal medium containing raffinose as a carbon source, then diluting and inoculating the precultured yeast cells into liquid minimal medium containing galactose and raffinose as the carbon source, and culturing it. Moreover, for example, when the host is Aspergillus sojae, and the expression control sequence is amyB promoter, the protein involved in the biological clock according to this invention can be produced, for example, by culturing in liquid minimal medium containing maltose as the carbon source. Moreover, for example, when the host is E. coli, and the expression control sequence is the lac promoter, the protein involved in the biological clock according to this invention can be produced by culturing in liquid medium containing IPTG. When the protein involved in the biological clock according to this invention is produced in the host cell or on the bacteria surface, the protein according to this invention can be obtained by isolating the host cell from the medium and then processing the cell appropriately. For example, when produced in the cell, the protein involved in the biological clock according to this invention can be isolated/purified by breaking up the cell physically or enzymatically, thereafter using centrifugation and various chromatographies and the like. When the protein involved in the biological clock according to this invention is produced in the culture medium, the protein according to this invention can be obtained using centrifugation/filtration and the like to remove bacterial bodies. In any case, using conventional methods such as ammonium sulfate fractionation, various chromatographies, alcohol precipitation, ultrafiltration and the like, the protein involved in the biological clock according to this invention can be purified to have even higher purity.

The protein involved in the biological clock according to this invention can be synthesized in vitro using an acellular protein synthesis system. The acellular protein synthesis system includes, for example, PROTEIOS (TOYOBO) (PROTEIOS (registered trademark)), which is the acellular protein synthesis system derived from wheat germ extract. Also, a part of the amino acid sequences of the protein involved in the biological clock according to this invention can be synthesized using in vitro artificial peptide synthesis. Such in vitro protein synthesis and peptide synthesis of the protein involved in the biological clock according to this invention is also included in this invention.

The protein involved in the biological clock according to this invention may be produced with addition of a tag sequence for using affinity chromatography during purification. Such a tag sequence includes, for example, GST tag, His tag, Myc tag and the like.

The present invention relates to the clock gene PHYTOCLOCK 1 (PCL1) which serves as the core of the biological clock of the higher plant, the protein PCL1 encoded by said gene, and the application of the PCL1 gene. In Arabidopsis wherein the PCL1 gene according to this invention was disrupted, all investigated circadian rhythms were lost, and photoperiodic flowering showed day-length insensitivity against the long-day nature of the wild type. Therefore, it is believed that it is possible to control various physiological phenomena/biological activity of the higher plant including photoperiodic flowering by manipulating artificially the PCL1 gene according to this invention.

### Example

Hereinafter, an example according to this invention will be described, but this invention is not intended to be interpreted with the restriction of the following example. The example as described below is only illustrated to describe one embodiment according to this invention, but this is not intended to exclude any modifications or alterations as long as it does not deviate from the spirit and the scope of this invention described in the accompanying claims.

### Example 1

The inventors screened comprehensively rhythm mutants which show anomaly in circadian rhythms using the bioluminescence rhythms of the GI::LUC⁺ luminescent reporter gene connecting the promoter of the GI gene of Arabidopsis to the coding region of the modified firefly luciferase gene (LUC⁺) as the indicator, as a result, had isolated five arrhythmic mutants. In these arrhythmic mutants, bioluminescence rhythms of the GI::LUC⁺ luminescent reporter gene in both constant light condition and constant dark condition were arrhythmic, and the nyctinasty of the leaves was also arrhythmic. Moreover, these arrhythmic mutations were all recessive single gene mutations, and could be classified into three complementary groups PHYTOCLOCK 1 (PCL1), PCL2, and PCL3 (Onai et al., Plant J., 41: 1-11 (2004)). One of the responsible genes of the arrhythmic mutation PCL1 gene was cloned using the map-based cloning method. The PCL1 gene satisfied all of the requirements mentioned above for the clock gene of the higher plant, so that it was concluded that it is the true clock gene of the plant, and that the protein encoded by the PCL1 gene is the clock protein of the plant. Homologous genes of the PCL1 gene also exist in rice, tobacco, tomato, potato, corn and pine, and it can be easily deduced that it serves as the clock gene in plants in general.

The present invention relates to the clock gene of the plant PCL1, and its homologous and similar genes which control oscillation and stabilization of circadian rhythms, more particularly the clock gene of plants PCL1 and its homologous genes as well as similar genes characterized by having a DNA biniding motif belonging to GARP family; DNA and RNA encoding said genes; a composition comprising the PCL1 protein, PCL1-homologous proteins or PCL1-similar proteins; a vector and a transformant that enable the expression of the PCL1 protein, PCL1-homologous proteins or PCL1-similar proteins; and a method for producing the PCL1 protein, PCL1-homologous proteins or PCL1-similar proteins.

Plant materials, culture conditions, and a method for measuring circadian rhythms used in the example

The bioluminescence reporter strain G-38 of Arabidopsis (Arabidopsis thaliana) carrying a GI::LUC⁺ luminescent reporter gene, which has an ecotype of Col-0, was used as the wild type strain unless otherwise indicated. Arrhythmic mutants pcl1-1 and pcl1-2 of Arabidopsis are arrhythmic mutants isolated from bioluminescence reporter strain G-38, and are mutants isolated as 23-15D9 and 32-5E2, respectively, in the report of the inventors (Onai et al., Plant J., 41: 1-11 (2004)). Homozygous plants of the fourth generation (F₄) of arrhythmic mutants pcl1-1 and pcl1-2 after backcrossing onto wild-type G-38 were used. Arabidopsis was grown aseptically on MS (Murashige and Skoog, Physiol. Plant. 15: 473-497 (1962)) solid medium containing 1.5% (w/v) sucrose according to the method of the inventors (Onai et al., Plant J., 41: 1-11 (2004). The illuminating light used during culturing of plants was white light of 70 μmol/m²/s.

### GI::LUC⁺ Bioluminescence of the pcl1 Mutant Under Constant Light Condition or Constant Dark Condition

In general, circadian rhythms continue autonomously under constant environments such as constant light and constant dark. Therefore, first, it was investigated whether GI::LUC⁺ bioluminescence of the pcl1 mutants oscillates autonomously under constant environment or not. The measurement of GI::LUC⁺ bioluminescence was performed according to the method of the inventors (Onai et al., Plant J., 41: 1-11 (2004)). After resetting the biological clock by giving the mutants a light/dark cycle of 12-hour light period/12-hour dark period or the cycle of 12-hour dark period/12-hour light period, bioluminescence was measured in constant light or constant dark.

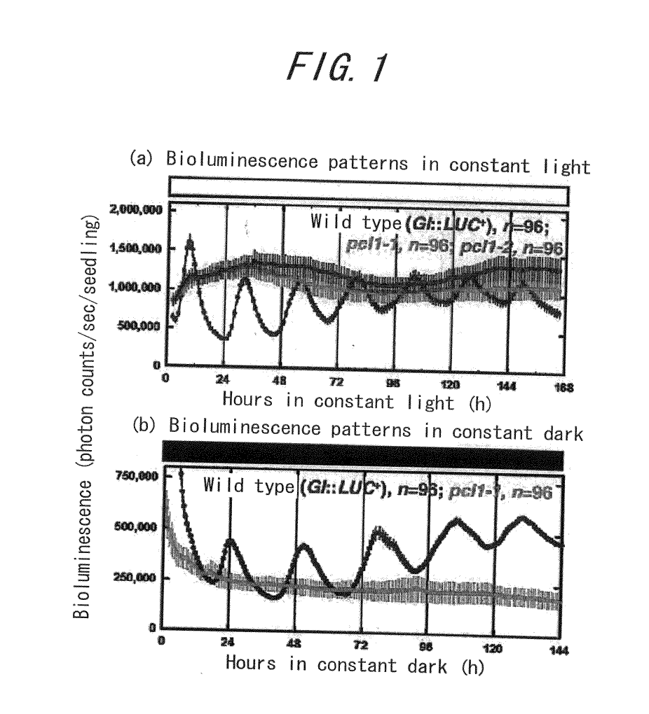

GI::LUC⁺ bioluminescence patterns of wild-type strain G-38 and the pcl1 mutants are shown in FIG. 1. Wild-type strain G-38 showed clear bioluminescence rhythms in both constant light and constant dark conditions, but the pcl1 mutants were arrhythmic in both conditions, and showed no rhythm. Therefore, it was found that the PCL1 gene is essential for the GI::LUC⁺ bioluminescence rhythms.

### Example 2

### GI::LUC⁺ Bioluminescence of the pcl1 Mutant Under Light/Dark Cycle Condition or Temperature Cycle Condition

In general, circadian rhythms are synchronized by light/dark cycle and temperature cycle. Therefore, it was investigated whether GI::LUC⁺ bioluminescence of wild-type strain G-38 and the pcl1 mutant are synchronized by light/dark cycle and temperature cycle. The measurement of GI::LUC⁺ bioluminescence under light/dark cycle and temperature cycle conditions was performed while giving the plants the light/dark cycle of 12-hour light period/12-hour dark period (the temperature during the light/dark cycle was constant at 22° C.) and the temperature cycle of 12-hour 22° C./12-hour 17° C. (under constant light during the temperature cycle), respectively.

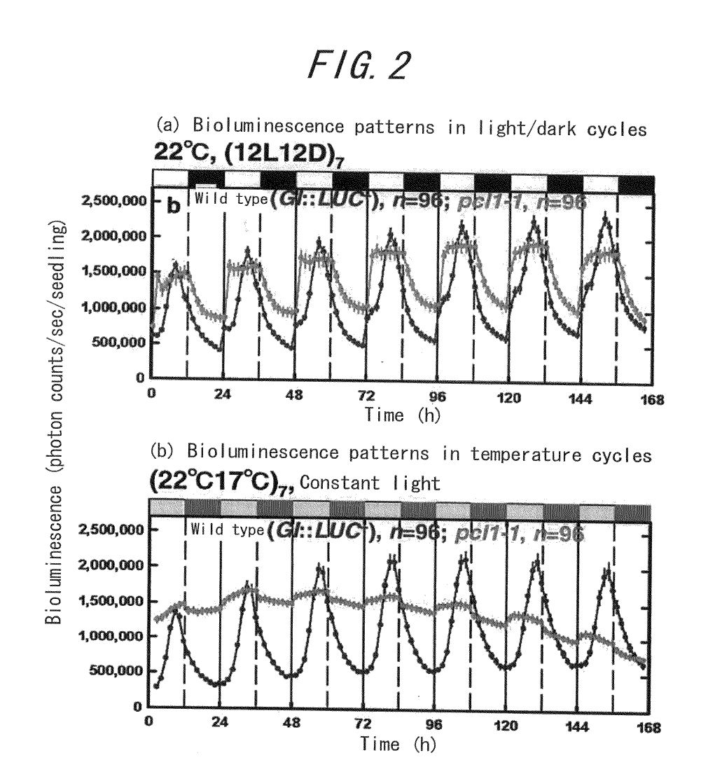

GI::LUC⁺ bioluminescence patterns of wild-type strain G-38 and the pcl1 mutant under light/dark cycle condition and GI::LUC⁺ bioluminescence patterns of wild-type strain G-38 and the pcl1 mutant under temperature cycle condition are shown in FIGS. 2a and 2b, respectively. Wild-type strain G-38 showed clear bioluminescence rhythms under light/dark cycle condition, while the bioluminescence pattern of the pcl1 mutant showed a rectangular waveform where in the light period, the amount of luminescence was kept high at a constant level, and in the dark period, the amount of luminescence decreased. This just reflects that GI gene is a light induced gene and the amount of expression is higher in light condition than in dark condition, and means that the bioluminescence of the pcl1 mutant shows no rhythm under light/dark condition. Similarly, wild-type strain G-38 also showed clear bioluminescence rhythms under temperature cycle condition, while the pcl1 mutant showed a rectangular luminescence waveform pattern where the amount of luminescence at 22° C. is slightly higher than that at 17° C. This just reflects that the enzymatic activity of the firefly luciferase used as the luminescent protein is slightly higher at 22° C. than at 17° C., and means that the bioluminescence of the pcl1 mutant shows no rhythm under temperature cycle condition. Therefore, it was found that the PCL1 gene is essential for the synchronization of the GI::LUC⁺ bioluminescence rhythms with light/dark cycle and temperature cycle.

### Example 3

### Nyctinasty of the Leaves of the pcl1 Mutant

In higher plants, it is widely known that the up-and-down motion (nyctinasty) of leaves show the circadian rhythms (Lumsden and Millar, Biological Rhythms and Photoperiodism in Plants. Oxford: Bios Scientific Publisher (1998)). If the aperiodism of the bioluminescence of the pcl1 mutant is caused by the anomaly of the main unit of the biological clock, it is expected that the nyctinastic rhythms of leaves, which is a different indicator from GI::LUC⁺ bioluminescence, is also arrhythmic. Therefore, the nyctinasty of the leaves of the pcl1 mutant was measured. The measurement of the nyctinasty of the leaves was performed according to the method of the inventors (Onai et al., Plant J., 41: 1-11 (2004)).

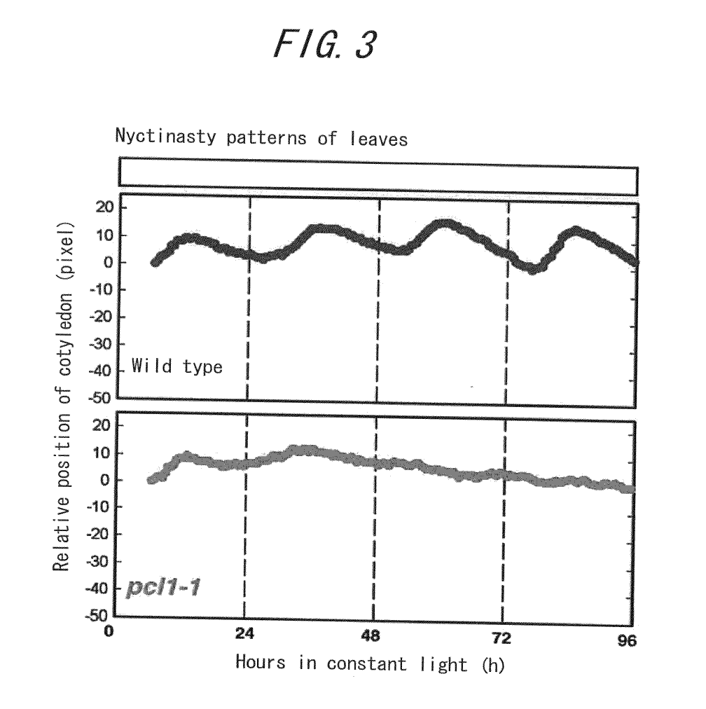

Nyctinasty patterns of wild-type strain and the pcl1 mutant are shown in FIG. 3. The wild-type strain showed clear nyctinastic rhythms, while the nyctinasty was not observed in the pcl1 mutant and was arrhythmic, and showed no rhythm. Therefore, it was found that the PCL1 gene is essential for the nyctinastic rhythms of leaves.

### Example 4

### Northern Blot Analysis of the Expression of GI, CAB2, TOC1, ELF4, CCA, and LHY Genes in the pcl1 Mutant

GI, TOC1, ELF4, CCA1 and LHY have been discovered as genes believed to be deeply involved in the biological clock of the higher plant, and the mRNA levels of these genes are known to show circadian rhythms (Young and Kay, Nat. Rev. Genet. 2: 702-715 (2001); Salomé and McClung, J. Biol. Rhythms 19: 425-435 (2004)). Also, the mRNA level of the CAB2 gene, which is a gene of the photosynthesis system, is known to show circadian rhythms (Millar and Kay, Science 267: 1161-1163 (1995)). In the pcl1 mutant, it was investigated using Northern blot analysis whether the circadian rhythms of the mRNA levels of these genes are disrupted or not. Northern blot analysis of the mRNA levels of the GI gene, CAB2 gene, TOC1 gene, ELF4 gene, CCA1 gene and LHY gene of wild-type strain G-38 and the pcl1-1 mutant were performed in the following procedure. First, the surface-sterilized seeds of wild-type strain G-38 and the pcl1-1 mutant were seeded on MS (Murashige and Skoog, Physiol. Plant. 15: 473-497 (1962)) solid medium containing 1.5% (w/v) sucrose, then they were cultured under conditions of constant light at 22.0±0.3° C. for 11 days, and after giving them 3 cycles of light/dark cycle of 12-hour light period/12-hour dark period, they were put back under constant light condition. The illuminating light during culturing of plants was white light of 50 μmol/m²/s. The end of the dark period of the 3^{rd} cycle, in other words the start of constant light, was taken to be the 0^{th} hour, and 10 plants were sampled and frozen immediately using liquid nitrogen at each time point of 3-hour intervals from the start of constant light. Then, total RNA from the frozen plants was extracted using RNeasy Midi Kit (RNeasy (registered trademark)) of QIAGEN. 5 μg of the total RNA was electrophoresed in 1.2% agarose gel containing formaldehyde, and then blotted onto a nylon film. The blotted total RNA was hybridized with ³²P-labeled gene-specific DNA probes, and the mRNA accumulation of each gene was detected as radioactivity. ³²P-labeled DNA probes specific to the genes TOC1, CCA1, and LHY1 were prepared according to the method of Makino et al. (Makino et al., Plant Cell Physiol. 43: 58-69 (2002)), respectively. The GI gene-specific ³²P-labeled DNA probe was prepared by cloning the base sequences from 8,064,660 to 8,066,052 registered at registration number NC_{—}003070 in GenBank/EMBL/DDBJ database from wild-type strain Col-0. The CAB2 gene-specific ³²P-labeled DNA probe was prepared by cloning the base sequences from 10,474,729 to 10,475,025 registered at registration number NC_{—}003070 in GenBank/EMBL/DDBJ database from wild-type strain Col-0. The ELF4 gene-specific ³²P-labeled DNA probe was prepared by cloning the base sequences from 16,741,294 to 16,742,019 registered at registration number NC_{—}003070 in GenBank/EMBL/DDBJ database from wild-type strain Col-0.

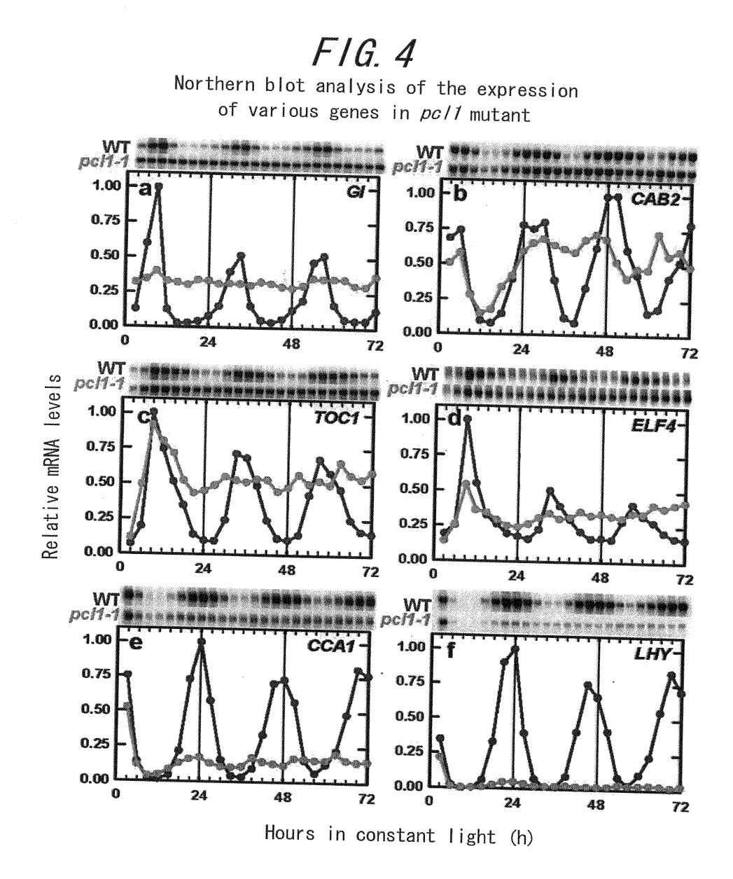

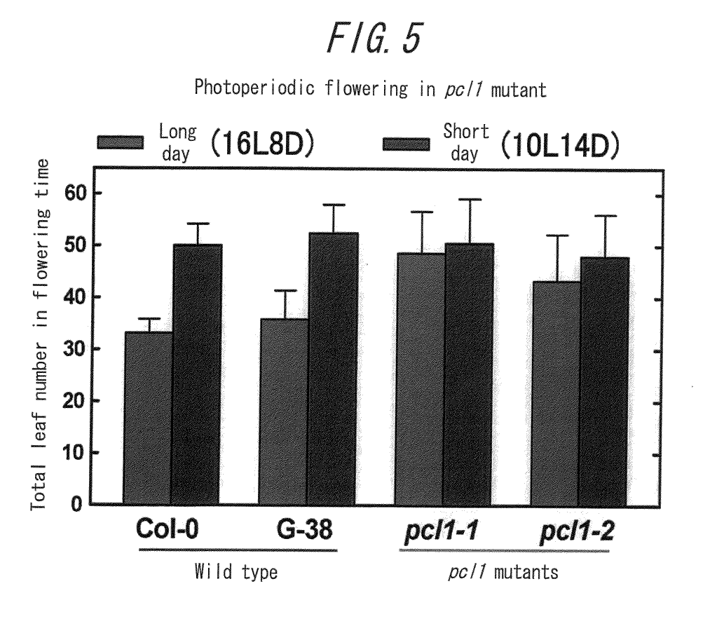

The results of Northern blot analysis are shown in FIG. 4. In wild-type strain G-38, the mRNA levels of each gene showed clear circadian rhythms. In contrast, in the pcl1 mutant, none of the mRNA levels showed the rhythms. Also, in the pcl1 mutant, the levels of GI mRNA, CAB2 mRNA, TOC1 mRNA and ELF4 mRNA were increased in comparison to the mRNA levels of the wild-type strain, and conversely, the levels of CCA1 mRNA and LHYmRNA were decreased significantly. These results show that the PCL1 gene is essential for rhythmic circadian expression of the GI gene, CAB2 gene, TOC1 gene and ELF4 gene, and that the PCL1 gene suppresses the expression of the GI gene, CAB2 gene, TOC1 gene and ELF4 gene and promotes the expression of the CCA1 gene and LHY gene.

### Example 5

### Photoperiodic Flowering of the pcl1 Mutant

The photoperiodic flowering of higher plants is known widely to be controlled by the biological clock (Sweeney, Rhythmic Phenomena in Plants 2^{nd} ed., Academic Press, San Diego (1987); Lumsden and Millar, Biological Rhythms and Photoperiodism in Plants. Oxford: Bios Scientific Publisher (1998)). It was investigated whether the photoperiodism is disrupted in the pcl1 mutant or not. The measurement of photoperiodic flowering was performed in the following procedure according to the method of Ohto et al. (Ohto et al., Plant Physiol. 127: 252-261 (2001)). After making the seeds of wild-type Col-0 strain and G-38 strain, and pcl1 mutants pcl1-1 and pcl1-2 absorb water, they were placed at 4° C. for 2 days in darkness, and then seeded on soil (vermiculite). Then they were cultured at 22.0±0.5° C. under long-day condition (16-hour light period/8-hour dark period) or short-day condition (10-hour light period/14-hour dark period). The illuminating light given to the plants during the light period was white light of 100 μmol/m²/s. The flowering time was quantified by counting the number of all the leaves of the plants when the plants bolted to the height of 1.5 cm. Arabidopsis is a long-day plant, and in the wild type, more leaves are formed when cultured under short-day condition in comparison to when cultured under long-day condition. The results of the measurement of the flowering time of wild-type strains Col-0 and G-38, and pcl1 mutants pcl1-1 and pcl1-2 are shown in FIG. 5. The wild-type strains showed a long-day nature of forming fewer leaves under long-day condition and more leaves under short-day condition. In contrast, the pcl1 mutants formed about the same number of leaves in both long-day and short-day day lengths, and were insensitive to day length. Therefore, the PCL1 gene was found to be essential for photoperiodic flowering.

### Example 6

### Map-Based Cloning of the PCL1 Gene and the Structure of the PCL1 Gene

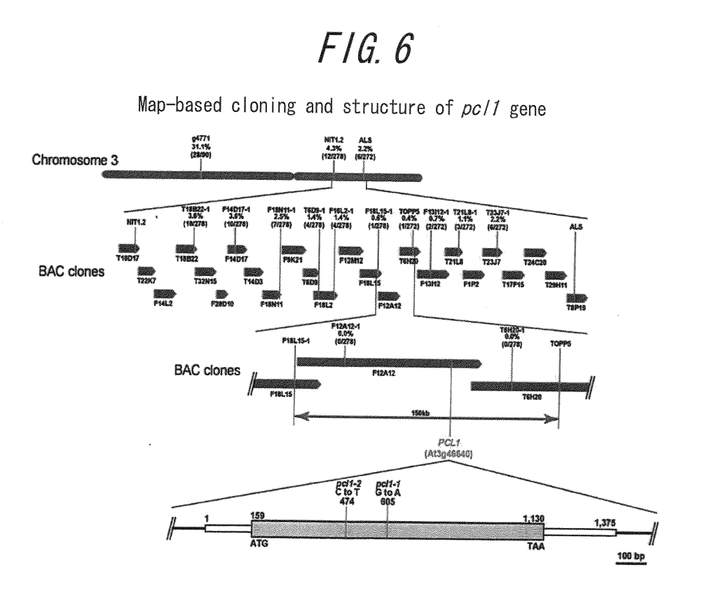

The cloning of the PCL1 gene was performed by the following procedure using the map-based cloning method. F₃ homozygotes (ecotype Col-0) of the pcl1-1 mutant and wild-type Ler strain were crossbred to obtain the 2^{nd} generation (F₂) seeds. The F₂ plants were cultured, the bioluminescence of the GI::LUC⁺ luminescence reporter gene was measured under constant light condition, and the homozygotes having the pcl1-1 mutation in homo were selected. Then, using the polymorphic markers (CAPS marker and SSLP marker) between Col-0 and Ler published at TAIR website (http://www.arabidopsis.org/) and SNP polymorphic markers released from Monsanto Arabidopsis Polymorphism Collection, the recombination rates between the pcl1-1 mutant and the polymorphic markers were calculated. Polymorphic markers having lower recombination rates are closer to the PCL1 gene physically, so the position of the PCL1 gene on the chromosomes was determined by examining the recombination ratio for various markers. In FIG. 6, the diagram of the map-based cloning of the PCL1 gene and the structure of the PCL1 gene are shown. The physical position of the PCL11 gene was determined to be at the area of about 150 kb between the SNP marker F18L15-1 (containing SNP numbers CER468139 to CER468143 of Monsanto Arabidopsis Polymorphism Collection) and the CAPS marker TOPP5 on the third chromosome. By determining and comparing the base sequences of wild-type Col-0 strain and G-38 strain, and arrhythmic mutants pcl1-1 and pcl1-2 in this area of about 150 kb, base substitution was found on the gene indexed at index number At3g46640 on the database at the TAIR website for both pcl1-1 and pcl1-2, so this was concluded to be the PCL1 gene. The structure of the PCL1 gene was determined by comparing the base sequence of the full-length cDNA and the genome DNA sequence published at RIKEN (RARGE; http://rarge.gsc.riken.go.jp/) and TAIR websites. The base sequence of the PCL1 gene and the deduced amino acid sequence of the PCL] protein are shown in SEQ ID No. 1 of the sequence listing. Both the pcl1-1 and pcl1-2 mutations were nonsense mutations generating stop codons in the coding region. The pcl1-1 mutation is a mutation wherein the 605^{th} base G in the base sequence shown in SEQ ID No. 1 of the sequence listing is substituted by A, and it can be found that the 149^{th} amino acid residue Trp in the amino acid sequence shown in SEQ ID No. 1 of the sequence listing becomes a stop codon. The pcl1-2 mutation is a mutation wherein the 474^{th} base C in the base sequence shown in SEQ ID No. 1 of the sequence listing is substituted by T, and it was found that the 106^{th} amino acid residue Gln in the amino acid sequence shown in SEQ ID No. 1 of the sequence listing becomes a stop codon.

### Example 7

### Structure of the PCL1 Protein

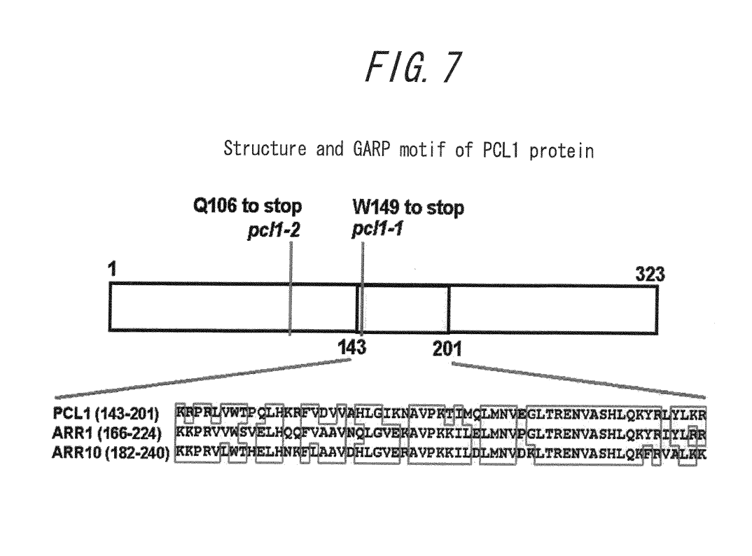

The deduced structure of the PCL1 protein is shown in FIG. 7. The PCL1 protein comprised 323 amino acid residues, and was a novel protein having no homology with known proteins. However, GARP motif, a DNA binding motif widely observed in the transcription factor of plants, was found in the central part (amino acid residues of numbers 143 to 201 of the amino acid sequence shown in SEQ ID No. 1 of the sequence listing) of the protein. The function and structure of the GARP motifs of the response regulators of Arabidopsis ARR1 protein (Sakai et al., Plant Cell Physiol. 39: 1232-1239 (1998)) and ARR10 protein (Imamura et al., Plant Cell Physiol. 40: 733-742 (1999)) had been investigated in detail. The ARR1 protein and ARR10 protein are known to localize in the nucleus and bind to certain DNA via the GARP motif. Furthermore, it is also known that the amino acid residues of the GARP motif of the ARR10 protein form a Myb-like helix-loop-helix structure, thereby binding to DNA (Hosoda et al., Plant Cell 14: 2015-2029 (2002)). Since the GARP motif in the PCL1 amino acid sequence is extremely similar to the GARP motifs of the ARR1 protein and ARR10 protein, it is suggested that the PCL1 protein localizes in the nucleus, binds to DNA via the GARP motif, and performs transcriptional control by binding to certain DNA. Since both pcl1-1 mutation and pcl1-2 mutation are recessive mutations and are nonsense mutations that form defective PCL1 proteins lacking the GARP motif, it is suggested that the both the pcl1-1 mutant and pcl1-2 mutant are PCL1-lacking mutants.

### Example 8

### Intracellular Localization of the PCL1 Protein

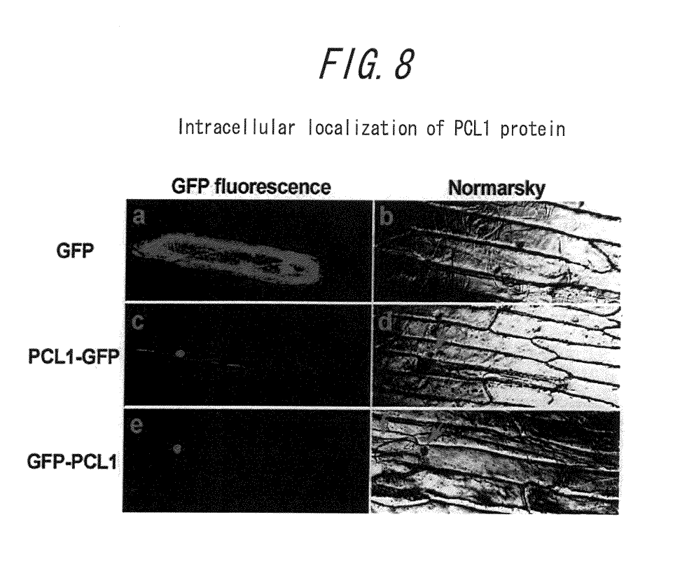

To investigate the intracellular localization of the PCL1 protein, referring to the method of Niwa et al. (Niwa et al., Plant J. 18: 455-463 (1999)), the GFP-PCL1 fusion protein and PCL1-GFP fusion protein were made to express transiently in onion epidermal cells, and the fluorescence of GFP was observed by the fluorescence microscope. For comparison, only GFP was made to express transiently in the onion epidermal cells, and the florescence of GFP was observed by the fluorescence microscope. As a result, the GFP-PCL1 fusion protein and the PCL1-GFP fusion protein localized in the nucleus (FIG. 8). Therefore, it is suggested that the PCL1 protein is localized in the nucleus inside the cell.

### Example 9

### Searching for PCL1-Similar Proteins and Homologous Proteins

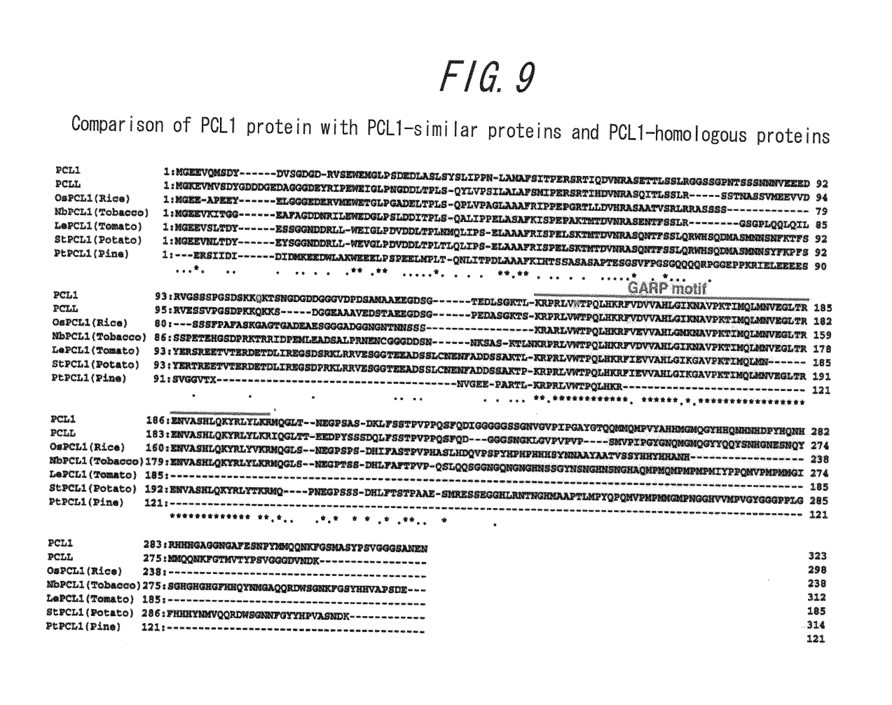

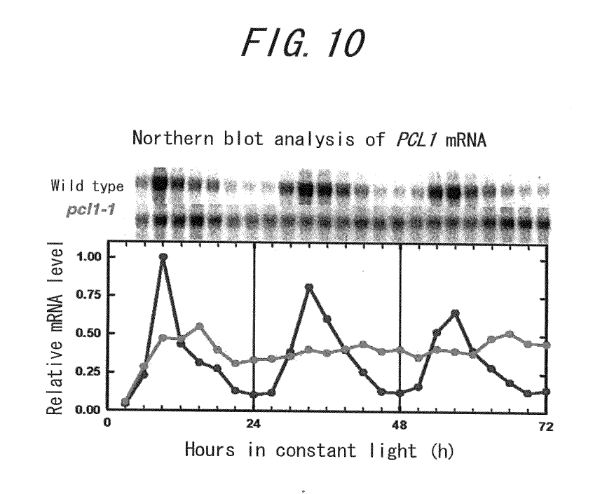

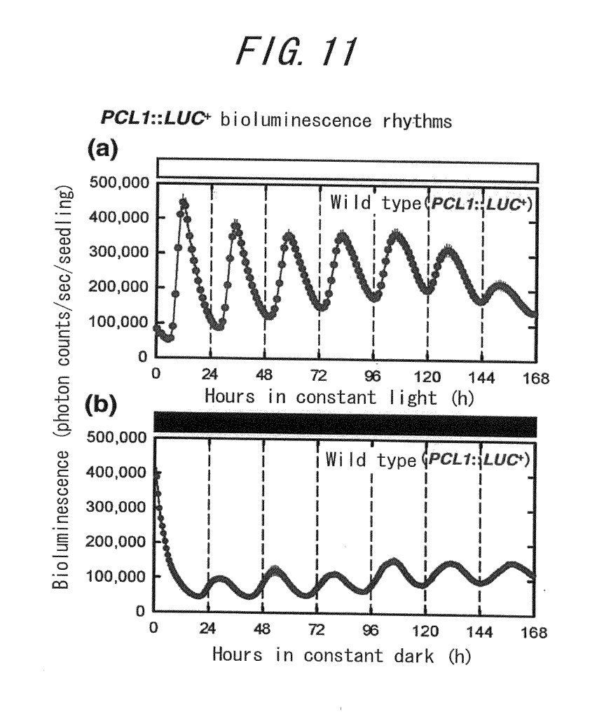

The homologous proteins and similar proteins of the PCL1 protein were searched for in public databases. As a result, genes that encode a PCL1-similar protein in Arabidopsis (Arabidopsis thaliana), genes that encode a PCL1-similar protein in rice (Oryza sativa), and cDNA that encode PCL1-homologous proteins in tobacco (Nicotiana benthamina and Nicotiana tabacum), tomato (Lycopersicon esculentum), potato (Solanum tuberosum), pine (Pinus taeda) and corn (Sorghum) were found, respectively. The result of the comparison of amino acid sequences of the PCL1 protein, PCL1-similar proteins and PCL1-homologous proteins are shown in FIG. 9. In Arabidopsis, the putative gene (the base sequence of SEQ ID No. 2 of the sequence listing) indexed in the database at TAIR website at index number At5g59570 encodes a protein (amino acid sequence of SEQ ID No. 9 of the sequence listing) having significant similarity with the PCL1 protein. The inventors named this gene PCL1-LIKE (PCLL). Furthermore, a putative gene (base sequence of SEQ ID No. 3 of the sequence listing) that encodes an amino acid sequence (amino acid sequence of SEQ ID No. 10 of the sequence listing) homologous with the PCL1 protein was found on rice genome DNA sequence, and the inventors named this gene the OsPCL1 gene. In tobacco (Nicotiana benthamina), cDNA (base sequence of base SEQ ID No. 4 of the sequence listing) deduced to encode a homologous protein (amino acid sequence of SEQ ID No. 11 of the sequence listing) of the PCL1 protein was found, although it is likely of being partial-length cDNA, and the inventors named this cDNA the NbPCL1 gene. In another species of tobacco (Nicotiana tabacum), cDNA (base sequence of SEQ ID No. 5 of the sequence listing) deduced to encode a homologous protein (amino acid sequence of SEQ ID No. 12 of the sequence listing) of the PCL1 protein was found, although it is partial-length cDNA, and the inventors named this cDNA the NtPCL1 gene. In tomato (Lycopersicon esculentum), cDNA (base sequence of SEQ ID No. 6 of the sequence listing) deduced to encode a homologous protein (amino acid sequence of SEQ ID No. 13 of the sequence listing) of the PCL1 protein was found, although it is partial-length cDNA, and the inventors named this cDNA the LePCL1 gene. In potato (Solanum tuberosum), cDNA (base sequence of SEQ ID No. 7 of the sequence listing) deduced to encode a homologous protein (amino acid sequence of SEQ ID No. 14 of the sequence listing) of the PCL1 protein was found, although it is partial-length cDNA, and the inventors named this cDNA the StPCL1 gene. In pine (Pinus taeda), cDNA deduced to encode a homologous protein (amino acid sequence of SEQ ID No. 15 of the sequence listing) of the PCL1 protein was found, although it is partial-length cDNA, and the inventors named this cDNA the PtPCL1 gene. In corn (Sorghum), cDNA deduced to encode a homologous protein of the PCL1 protein (amino acid sequence of SEQ ID No. 16 of the sequence listing) was found, although it is partial-length cDNA.

### Example 10

### Northern Blot Analysis of the Expression of the PCL1 Gene

In biological species wherein clock genes have been found up to today, the expression of almost all clock genes shows circadian rhythms (Ishiura et al., Science 281: 1519-1523; Dunlap, Cell 96: 271-290). Therefore, it was investigated using Northern blot analysis whether the expression of the PCL1 gene shows circadian rhythms or not. Northern blot analysis of the PCL1 mRNA levels in the cells of wild-type strain G-38 and the pcl1 mutant was performed in the following procedure. First, the surface-sterilized seeds of wild-type strain G-38 and the pcl1-1 mutant were seeded on MS (Murashige and Skoog, Physiol. Plant. 15: 473-497 (1962)) solid medium containing 1.5% (w/v) sucrose, then they were cultured under conditions of constant light at 22.0±0.3° C. for 11 days, and after giving them 3 cycles of light/dark cycle of 12-hour light period/12-hour dark period, they were put back under constant light condition. The illuminating light during culturing of plants was white light of 50 μmol/m²/s. The end of the dark period of the 3^{rd} cycle, in other words the start of constant light, was taken to be the 0^{th} hour, and 10 plants were sampled and frozen immediately using liquid nitrogen at each time point of 3-hour intervals from the start of constant light. Then total RNA from the frozen plants was extracted using RNeasy Midi Kit of QIAGEN. 5 μg of the total RNA was electrophoresed in 1.2% agarose gel containing formaldehyde, and then blotted onto a nylon film. The blotted total RNA was hybridized with ³²P-labeled PCL1 gene-specific DNA probe, and the mRNA accumulation of the PCL1 gene was detected as radioactivity. The PCL1 gene-specific ³²P-labeled DNA probe was prepared by cloning 1,021 to 1,992 of the base sequence shown in SEQ ID No. 1 of the sequence listing from wild-type strain Col-0.

The results of Northern blot analysis is shown in FIG. 10. In wild-type strain G-38, the mRNA level of the PCL1 gene showed clear circadian rhythms having a peak in the subjective early evening. In contrast, in the pcl1 mutant, the PCL1 mRNA level showed no circadian rhythm. Therefore, it was found that the expression of the PCL1 gene shows circadian rhythms, and is essential for its own rhythmic circadian expression.

### Example 11

### Construction and Bioluminescence Rhythms of PCL1::LUC⁺ Bioluminescence Reporter Strain

In order to monitor in real time the expression of the PCL1 gene as bioluminescence in detail, the PCL1::LUC⁺ bioluminescence reporter strain was constructed in the following procedure. First, a bioluminescence reporter gene cassette (PCL1::LUC⁺) connecting the promoter region (1 to 1,020 of the base sequence shown in SEQ ID No. 1 of the sequence listing) of the PCL1 gene to the coding region of modified firefly luciferase gene (LUC⁺; Promega) was constructed, and this cassette was inserted into the cloning site of binary vector pBIB-Hyg (Becker, Nucleic Acids Res. 18: 203 (1990)) to construct pBIB/PCL1::LUC⁺. According to the method of Clough and Bent (Clough and Bent, Plant J. 16: 735-743 (1998)), the T-DNA region of pBIB/PCL1::LUC⁺ (which contains hygromycin B-resistant gene HPT and PCL1::LUC⁺) was transferred via agrobacteria into the genome of Arabidopsis wild-type strain Col-0. The transformants obtained by gene transfer was selected as hygromycin-B resistant plants according to the description of Weigel and Glazebrook (Weigel and Glazebrook, ARABIDOPSIS: A Laboratory Manual Cold Spring Harbor Laboratory Press, Cold Spring Harbor (2002)), and then homozygotes (T₃) inserting T-DNA into a single genetic locus was selected and used as wild-type PCL1::LUC⁺ bioluminescence reporter strain.

The measurement of PCL1::LUC⁺ bioluminescence was performed according to the method of the inventors (Onai et al., Plant J., 41: 1-11 (2004)). After resetting the biological clock by giving it the light/dark cycle of 12-hour light period/12-hour dark period or the cycle of 12-hour dark period/12-hour light period, bioluminescence was measured under constant light or constant dark.

The bioluminescence pattern of wild-type PCL1::LUC⁺ luminescence reporter strain is shown in FIG. 11. The bioluminescence of wild-type PCL1::LUC⁺ luminescence reporter strain under constant light condition showed clear circadian rhythms matching the circadian rhythms of PCL1 mRNA from Northern blot analysis. Furthermore, although the amount of bioluminescence decreased to about one thirds compared to the case of constant light, the bioluminescence of wild-type PCL1::LUC⁺ luminescence reporter strain showed clear circadian rhythms also under constant dark condition. These results mean that the expression of the PCL1 gene shows clear circadian rhythms under both constant light and constant dark conditions.

### Example 12

### Construction of PCL1-Overexpressing-Plants PCL1-ox and the GI::LUC⁺ Bioluminescence Pattern and the Leaves Nyctinasty of the PCL1-ox Plants

In biological species wherein clock genes have been found up to today, it is widely known that the expression of almost all clock genes shows circadian rhythms, and when these circadian rhythms are disrupted, all circadian rhythms disappears (Ishiura et al., Science 281: 1519-1523; Dunlap, Cell 96: 271-290). Therefore, it was investigated whether the circadian rhythms disappear or not by constructing a PCL1-overexpressing-plants PCL1-ox and destroying the circadian expression of the PCL1 gene. The PCL1-overexpressing-plants PCL1-ox was constructed in the following procedure. First, the PCL1 overexpression cassette (CaMV35S::PCL1) connecting the 35S promoter (CaMV35S; base sequence of 4,951 to 5,815 registered at registration number AF485783 in the GenBank/EMBL/DDBJ database) of cauliflower mosaic virus to the coding region of the PCL1 gene (base sequence of 997 to 2,001 shown in SEQ ID No. 1 of the sequence listing) was constructed, and this cassette was inserted into the cloning site of binary vector pBIB-Hyg (Becker, Nucleic Acids Res. 18: 203 (1990)) to construct pBIB/35S::PCL1. According to the method of Clough and Bent (Clough and Bent, Plant J. 16: 735-743 (1998)), the T-DNA region of pBIB/35S::PCL1 (which contains hygromycin B-resistant gene HPT and 35S::PCL1) was transferred via agrobacteria into the genome of Arabidopsis wild-type strain G-38 (the wild-type strain that has the GI::LUC⁺ luminescence reporter gene). The transformants obtained by gene transfer was selected as hygromycin-B resistant plants according to the description of Weigel and Glazebrook (Weigel and Glazebrook, ARABIDOPSIS: A Laboratory Manual Cold Spring Harbor Laboratory Press, Cold Spring Harbor (2002)), and then homozygotes (T₃) inserting T-DNA into a single genetic locus was selected, and after confirming that the PCL1 mRNA level in the cell was increased using Northern blot analysis, this was used as the PCL1-overexpressing-plants PCL1-ox.

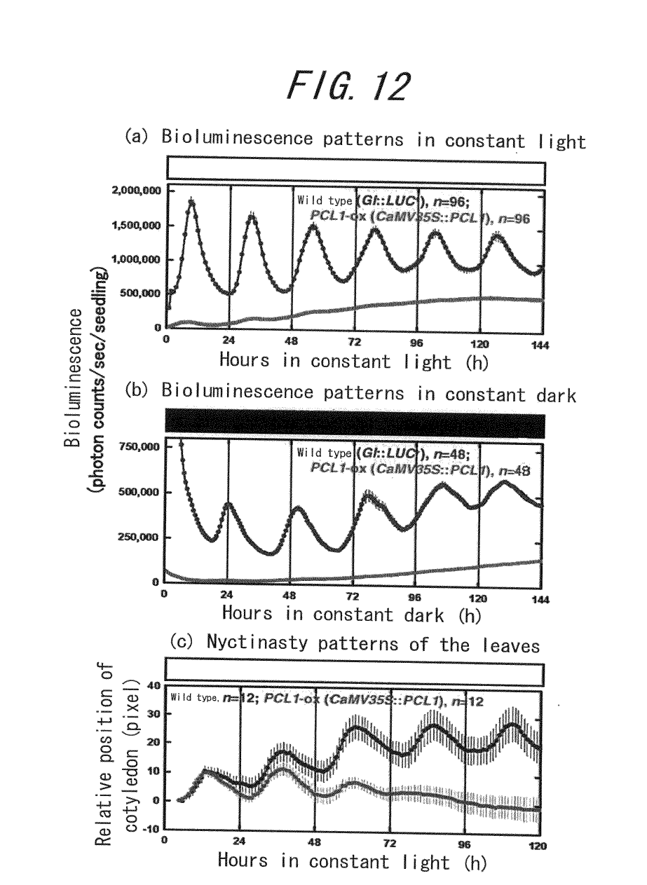

The GI::LUC⁺ bioluminescence patterns of wild-type strain G-38 and PCL1-ox plants under constant light and constant dark conditions are shown in FIG. 12a and FIG. 12b. The measurement of GI::LUC⁺ bioluminescence was performed according to the method of the inventors (Onai et al., Plant J., 41: 1-11 (2004)). After resetting the biological clock by giving it the light cycle of 12-hour light period/12-hour dark period or the cycle of 12-hour dark period/12-hour light period, bioluminescence was measured under constant light or constant dark. Wild-type strain G-38 showed clear bioluminescence rhythms under both constant light and constant dark conditions, but in PCL1-ox plants, the rhythms disappeared in both conditions by the 3^{rd}-4^{th} day. Also, in the PCL1-ox plants, the GI::LUC⁺ bioluminescence level was constantly lower than wild-type strain G-38. That is, the overexpression of the PCL1 gene disrupted the GI::LUC⁺ bioluminescence rhythms, and suppressed the expression of the GI::LUC⁺ reporter gene.

Nyctinasty patterns of the leaves of the wild-type strain and the PCL1-ox plants are shown in FIG. 12c. The measurement of the nyctinasty of the leaves was performed according to the method of the inventors (Onai et al., Plant J., 41: 1-11 (2004)). The wild-type strain showed clear nyctinastic rhythms, but in the PCL1-ox plants, the nyctinastic rhythms disappeared by the 4^{th} day. That is, the overexpression of the PCL1 gene also disrupted the nyctinastic rhythms of the leaves.

These results show that the circadian rhythms disappear when the circadian expression of the PCL1 gene is disrupted. That is, rhythmic circadian expression of the PCL1 gene is essential for circadian rhythm oscillation.

### Example 13

### The Self-Feedback Control of the Expression of the PCL1 Gene

In biological species wherein clock genes have been discovered up to today, the expression of the clock gene controls the gene expression of itself, in other words controls itself through self-feedback, and this makes rhythmic circadian expression possible. And this self-feedback loop is known widely to be the essence of clock oscillation (Ishiura et al., Science 281: 1519-1523; Dunlap, Cell 96: 271-290). For example, Period gene of Drosophila and mammals, frequency gene of Neurospora, and kaiC gene of cyanobacteria controls the gene expression of itself through negative feedback. Therefore, in order to demonstrate whether the expression of the PCL1 gene is controlled through self-feedback or not, the expression of the endogenous PCL1 gene in the PCL1-ox plants (plants carrying the CaMV35S::PCL1 gene which is the PCL1 gene overexpression cassette in an ectopic part on the chromosomes in addition to the endogenous PCL1 gene) was investigated using Northern blot analysis. Northern blot analysis of wild-type strain G-38, the pcl1 mutant and the PCL1-ox plants were performed in the following procedure. First, the surface-sterilized seeds of these were seeded respectively on MS (Murashige and Skoog, Physiol. Plant. 15: 473-497 (1962)) solid medium containing 1.5% (w/v) sucrose, then they were cultured under conditions of constant light at 22.0±0.3° C. for 11 days, and after giving them 3 cycles of light/dark cycle of 12-hour light period/12-hour dark period, they were put back under constant light condition. The illuminating light during plants culturing was white light of 50 μmol/m²/s. The end of the dark period of the 3^{rd} cycle, in other words the start of constant light, was taken to be the 0^{th} hour, and 10 plants were sampled and frozen immediately using liquid nitrogen at each time point of 4-hour intervals from the start of constant light. Then, total RNA from the frozen plants was extracted using RNeasy Midi Kit of QIAGEN. 5 μg of total RNA was electrophoresed in 1.2% agarose gel containing formaldehyde and then blotted onto a nylon film. Total RNA that was blotted was hybridized with an RNA probe that hybridizes only with mRNA transcripted from the endogenous PCL1 gene, and the mRNA accumulation derived from the endogenous PCL1 gene was detected as radioactivity. For the RNA probe that hybridizes specifically with mRNA derived from the endogenous PCL1 gene, the 3′ nontranslated region (base sequence of 1,992 to 2,237 shown in SEQ ID No. 1 of the sequence listing) of PCL1 mRNA synthesized in vitro while taking in ³²P was used.

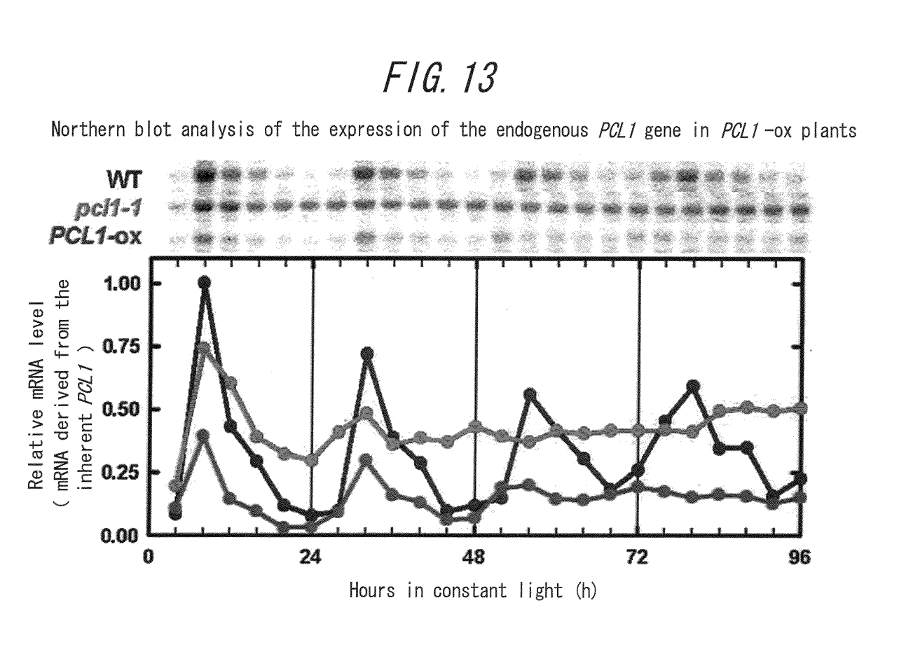

The results of Northern blot analysis are shown in FIG. 13. In wild-type strain G-38, the PCL1 mRNA fluctuated rhythmically and showed clear circadian rhythms, but in the PCL1-ox plants, the circadian rhythms of the level of mRNA transcribed from the endogenous PCL1 gene disappeared at the 3^{rd} day, and its level was lower compared to the wild-type. Also, in the pcl1 mutant, the PCL1 mRNA level was higher compared to the wild type, and showed no circadian rhythm. These results show that the PCL1 gene expression controls the gene expression of itself negatively, in other words, the negative PCL1 gene expression control forms a self-feedback loop, and this feedback loop is essential for circadian rhythm oscillation.

From the above results, it has been found that the following five points that are the requirements for the biological clock gene in the higher plant mentioned above, that is, (1) all circadian rhythms are lost due to the loss of function of one gene, (2) photoperiodism is lost due to the loss of function of one gene, (3) gene expression shows circadian rhythms under constant light and constant dark conditions, (4) all circadian rhythms are disrupted by the overexpression of the gene, (5) the gene controls the expression of itself through feedback control, are satisfied by the gene of this invention. The gene that satisfies these conditions had not been found until the making of this invention as mentioned above.

In Example 6, the inventors cloned the biological clock gene PCL1, which controls circadian rhythms from Arabidopsis genome. Furthermore, in Examples 1-4 and Example 10, all the circadian rhythms investigated in the pcl1 mutant were lost. Therefore, in the PCL1 gene, all circadian rhythms are lost due to the loss of function of one gene, so it was demonstrated that the PCL1 gene satisfies the above requirement (1).

In Example 5, photoperiodic flowering was day-length insensitive in the pcl1 mutant. Therefore, in the PCL1 gene, photoperiodism is lost due to the loss of function of one gene, so it was demonstrated that the PCL1 gene satisfies the above requirement (2).

In Example 10 and Example 11, the expression of the PCL1 gene showed circadian rhythms under constant light and constant dark conditions. Therefore, it was demonstrated that the PCL1 gene satisfies the above requirement (3).

In Example 12 and Example 13, the overexpression of the PCL1 gene disrupted all the investigated circadian rhythms. Therefore, it was found that the PCL1 gene satisfies the above requirement (4).

In Example 13, the loss of function of the PCL1 gene increased the expression of itself, and the overexpression suppressed the expression of itself. Therefore, the PCL1 gene controls the expression of itself through negative self-feedback, so it was demonstrated that the PCL1 gene satisfies the above requirement (5).

From the above, the PCL1 gene satisfies all the requirements for the biological clock gene, and was demonstrated to be the clock gene of plants.

Moreover, in Example 7 and Example 8, it was found that the PCL1 gene encodes nucleus-localized proteins having GARP motifs. Therefore, the PCL1 protein is suggested to be localized in the nucleus of plant cells and functions as a transcription factor that binds to certain DNA and controls gene expression.

In Example 9, the genes that encode similar proteins or homologous proteins of the PCL1 protein were found in many plants. These proteins are easily assumed to function as the clock protein.

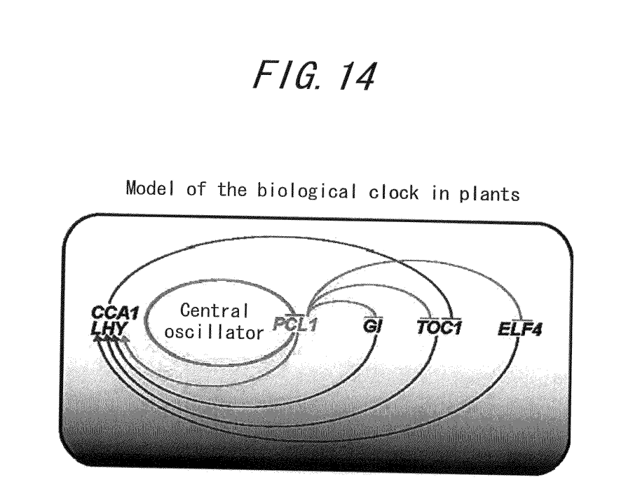

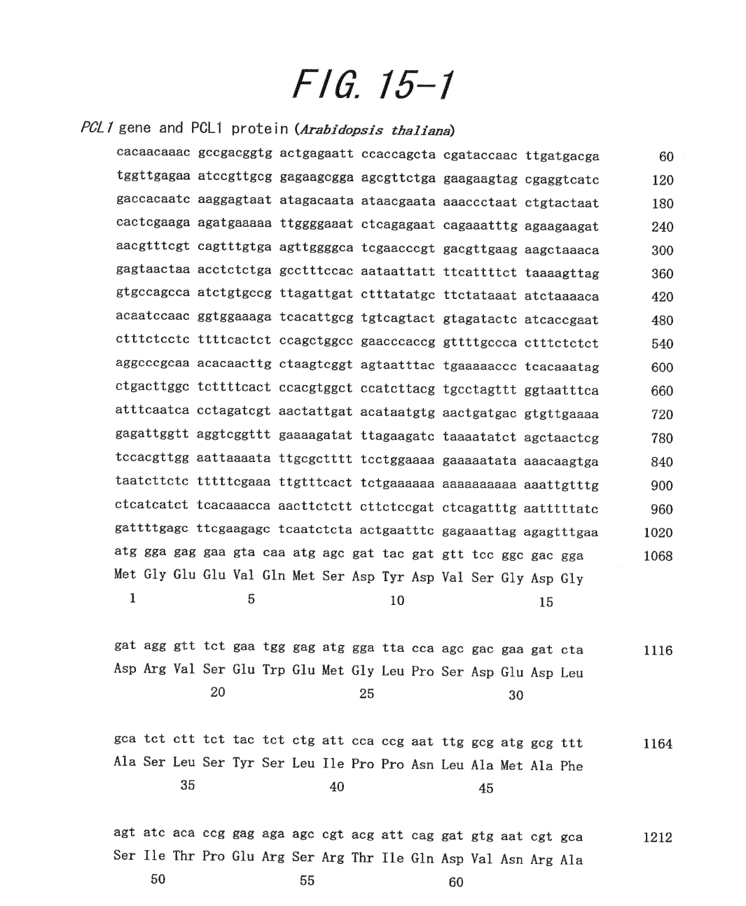

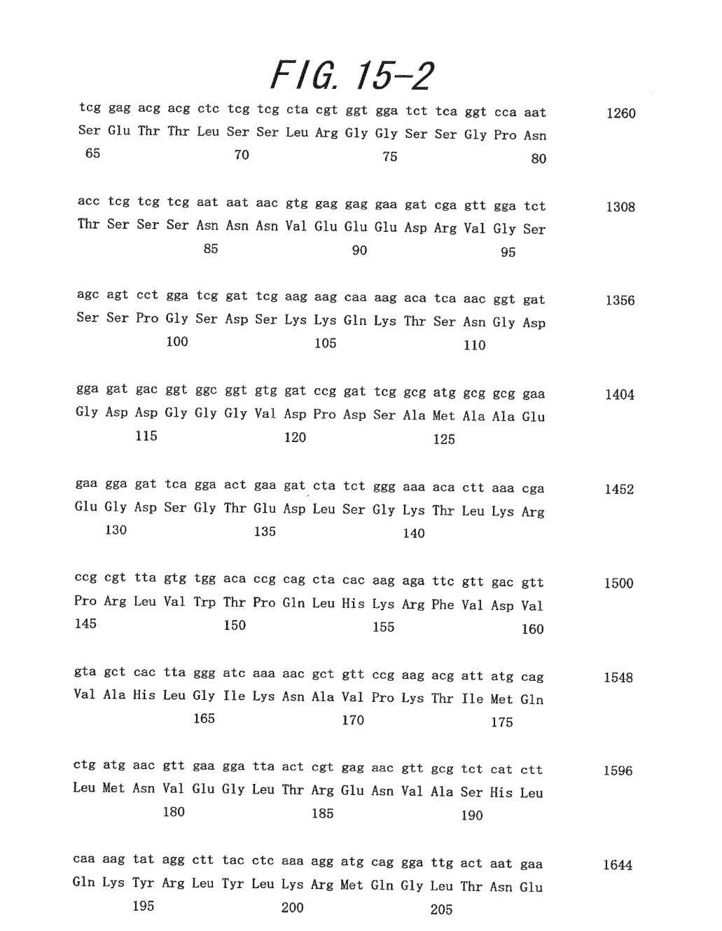

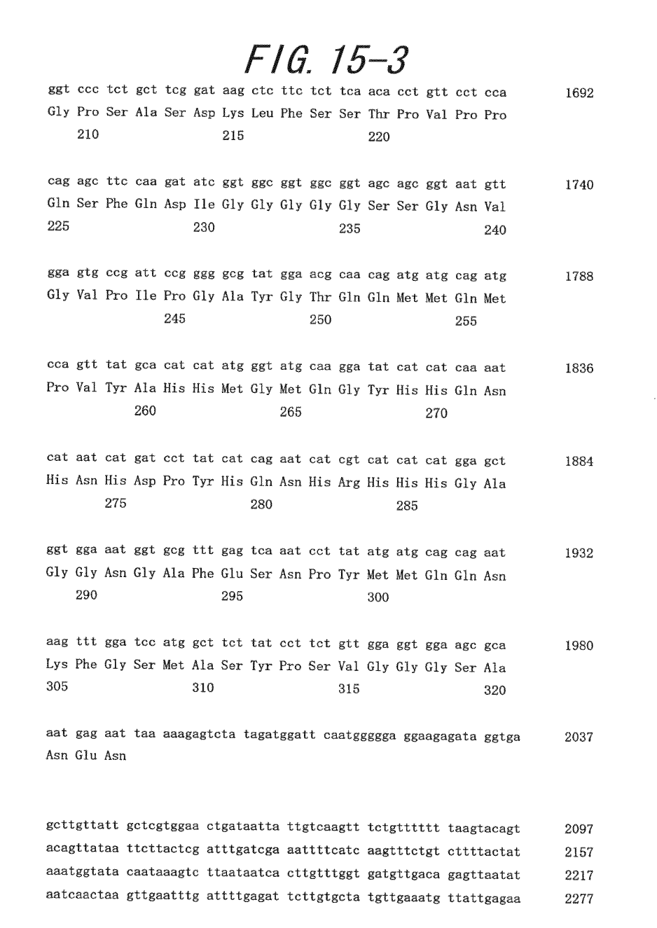

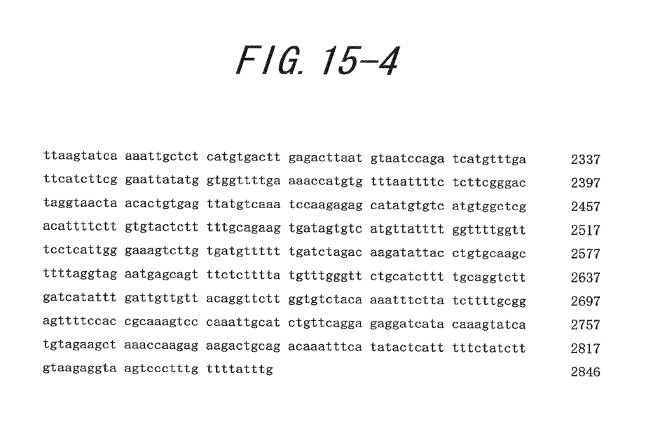

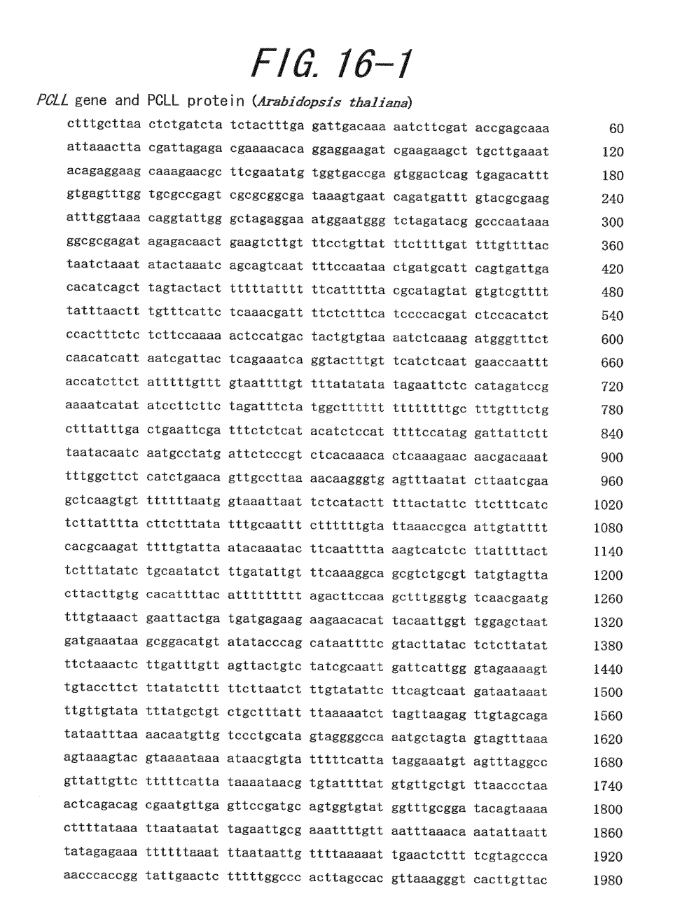

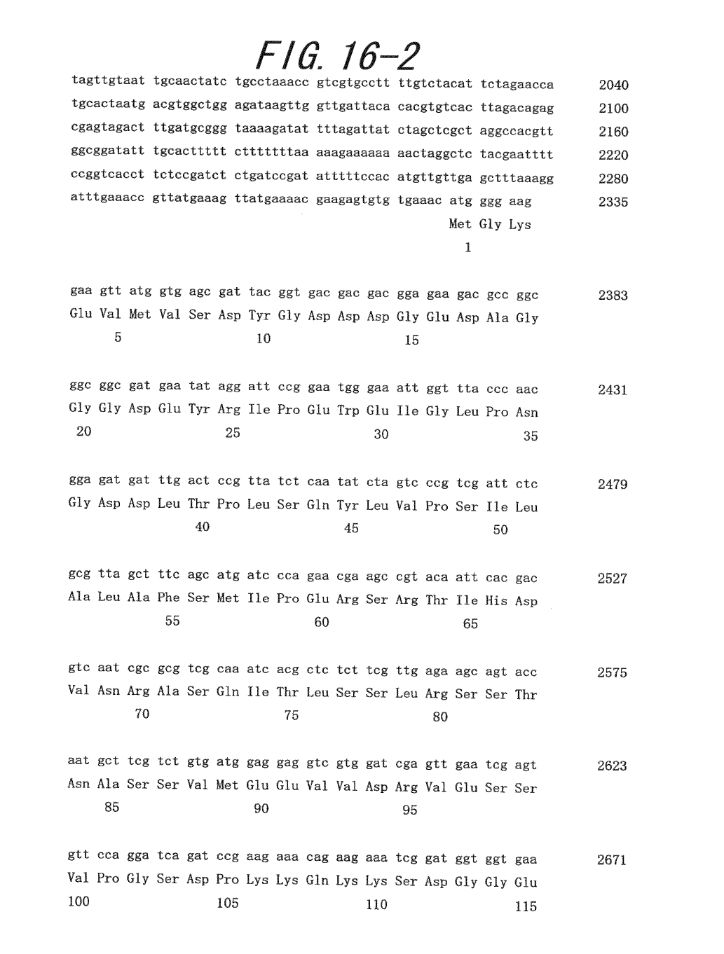

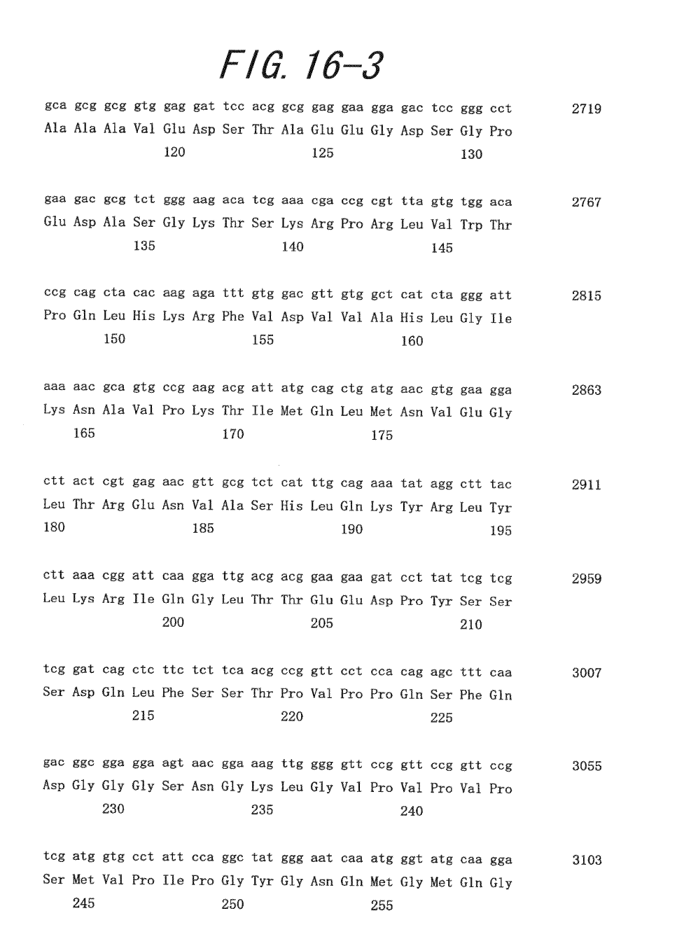

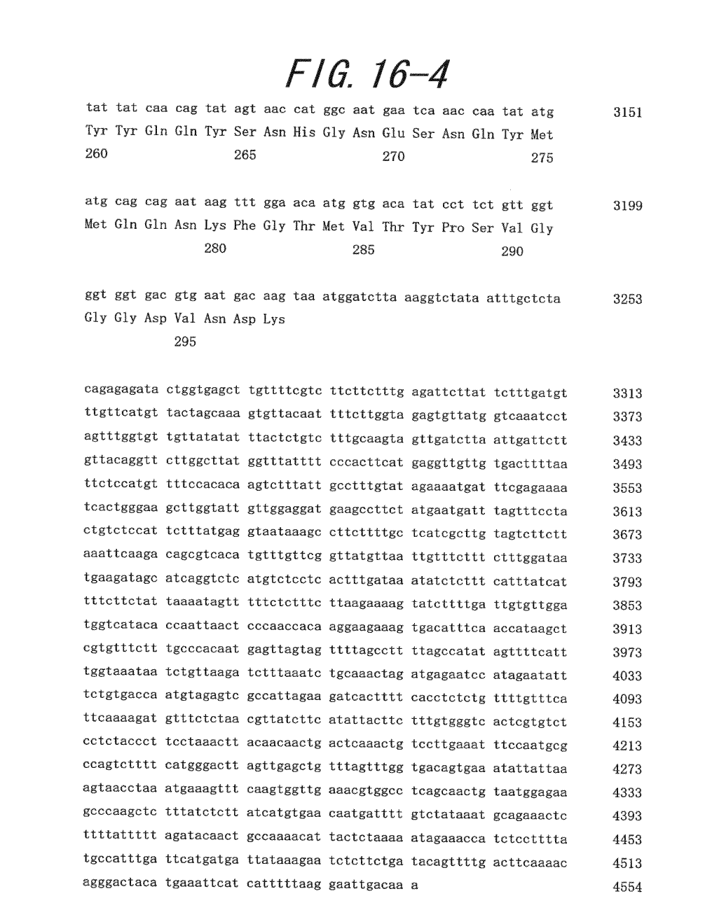

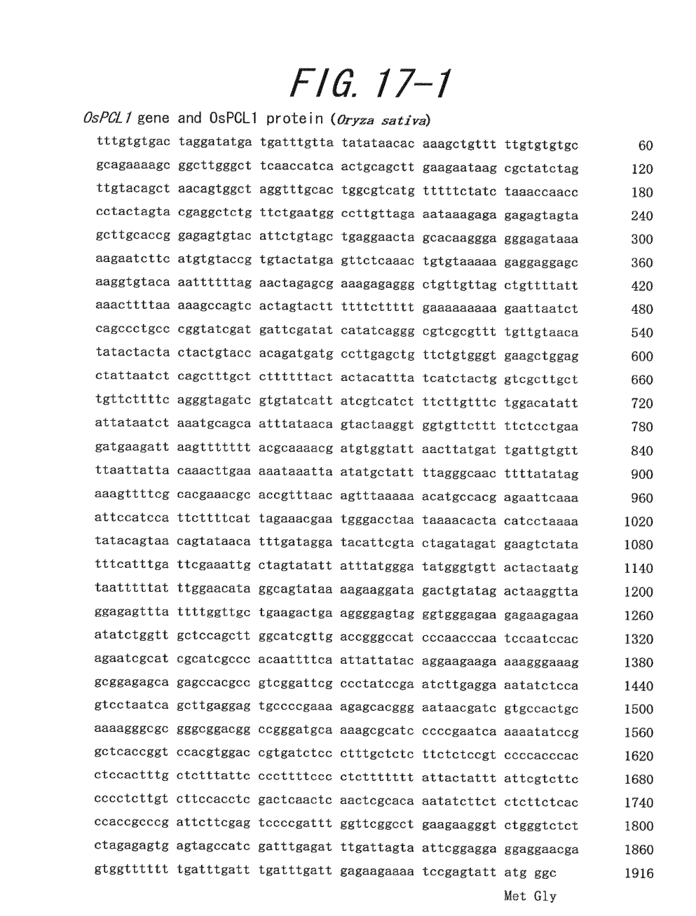

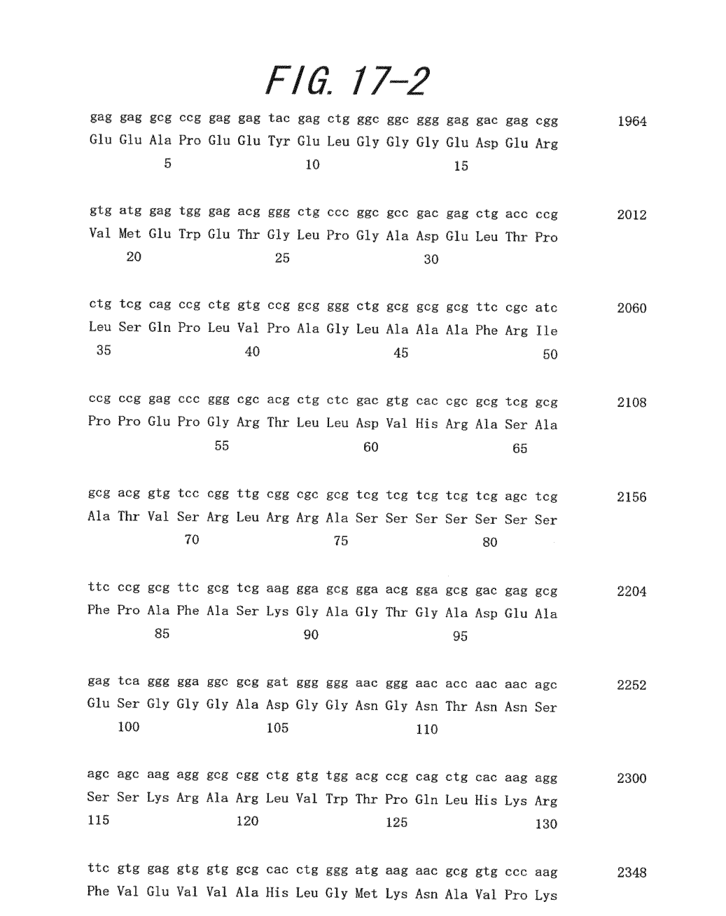

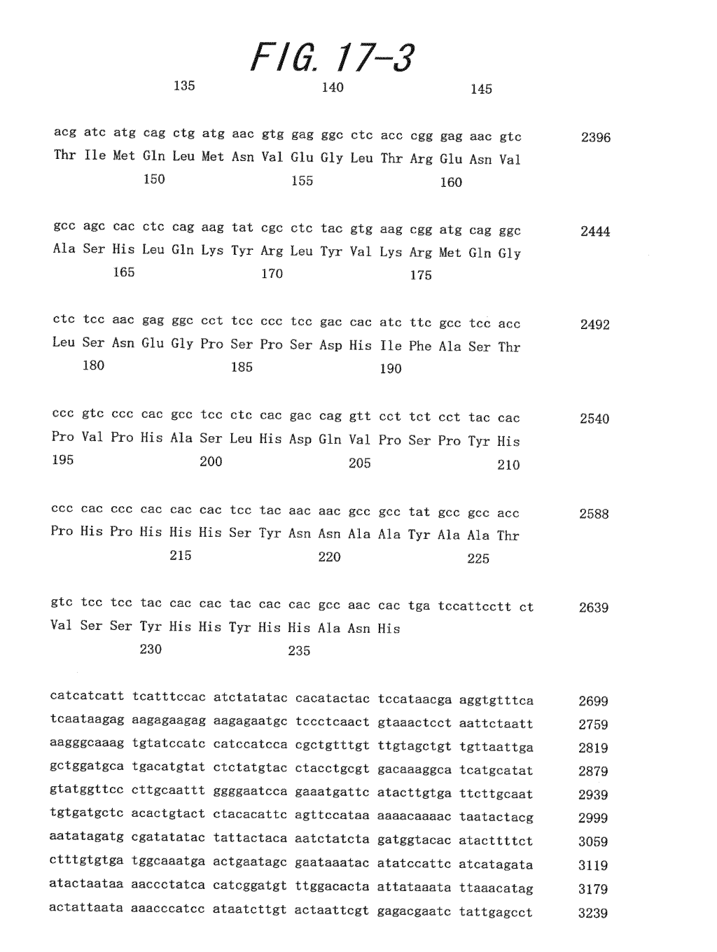

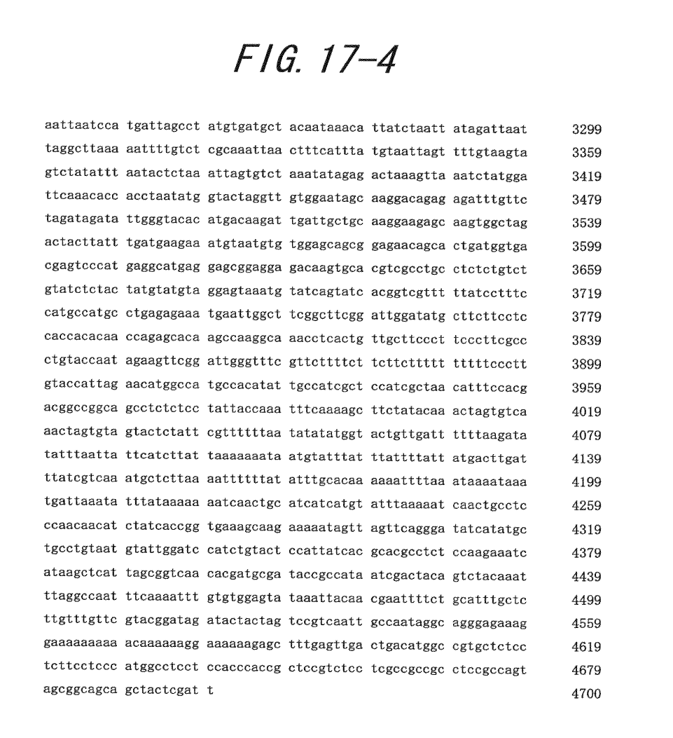

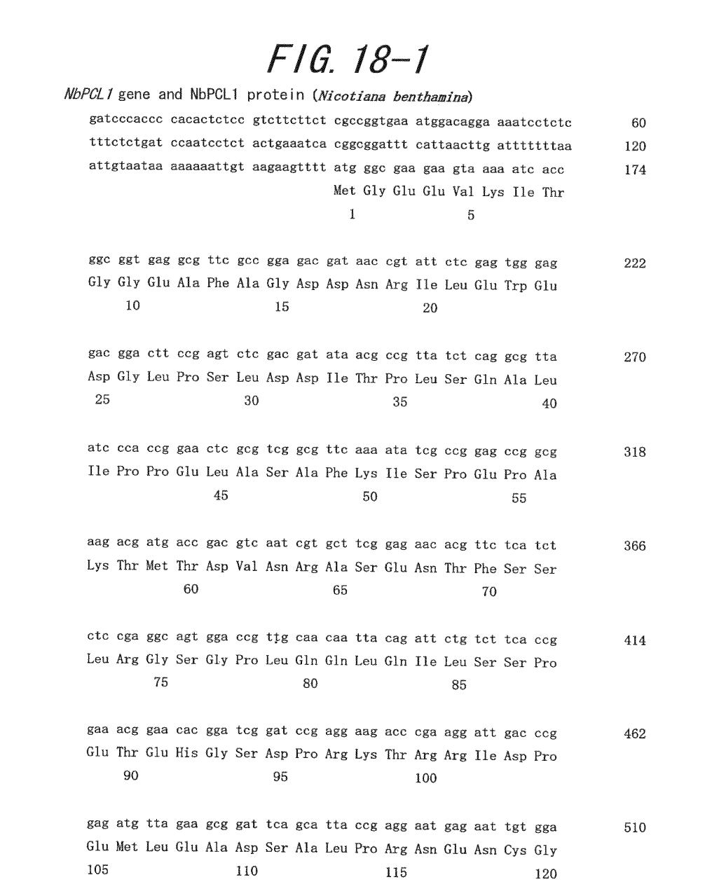

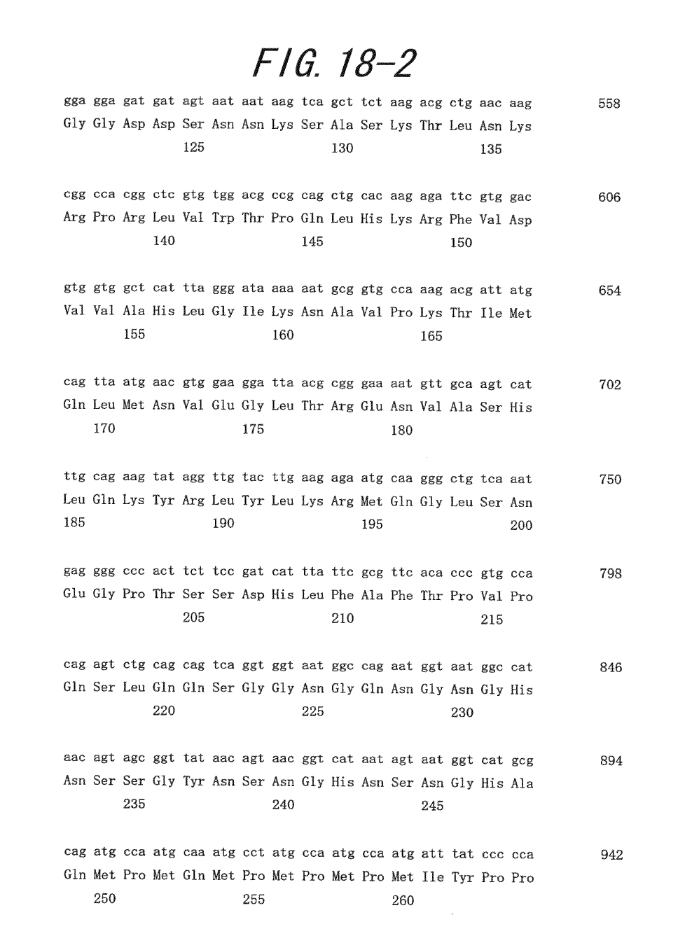

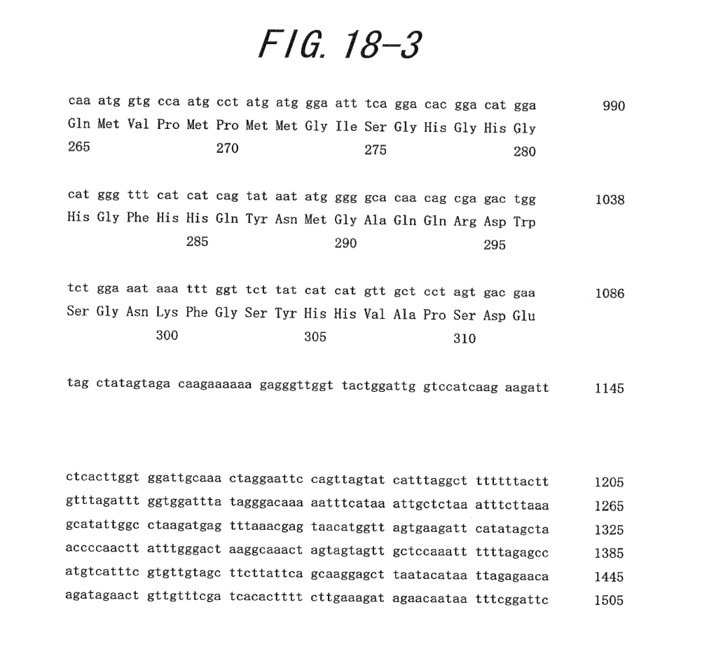

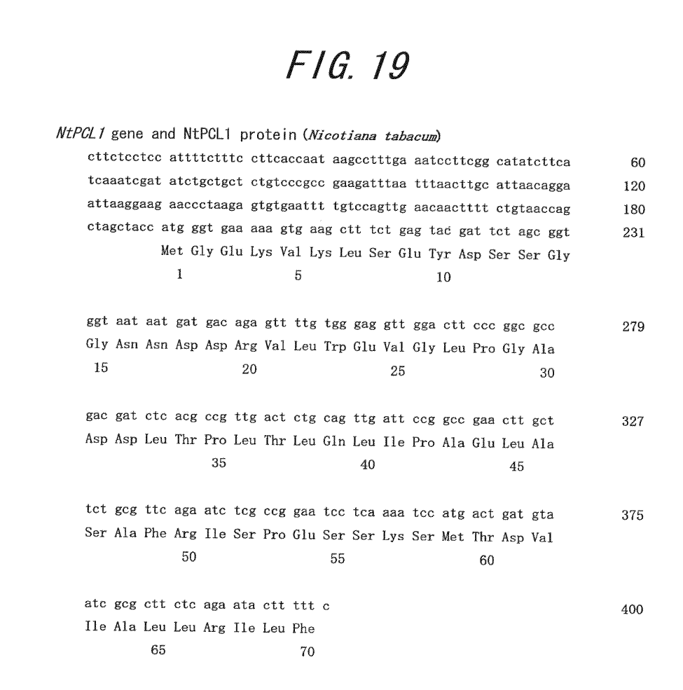

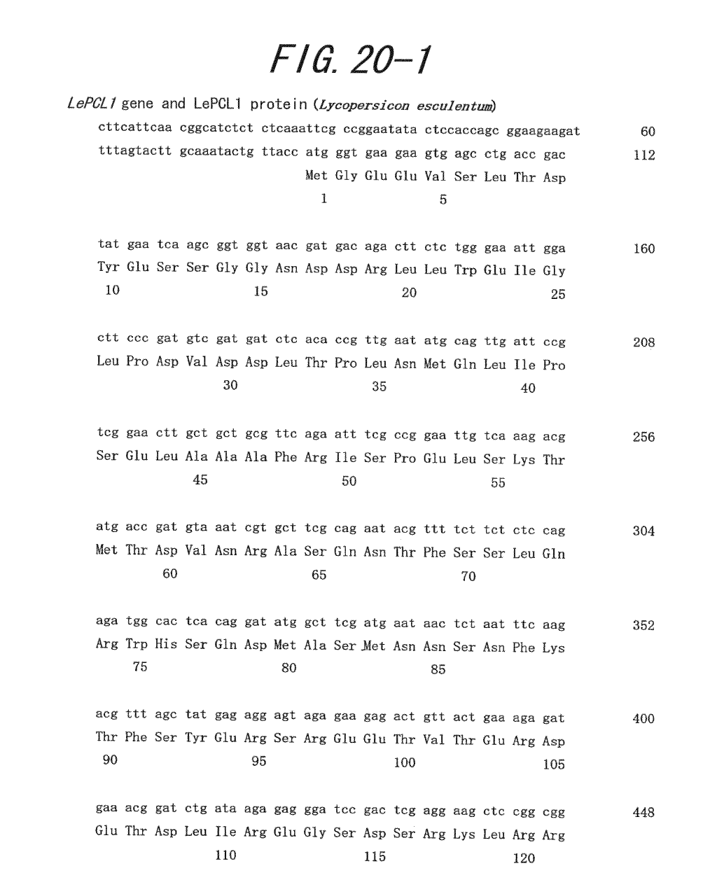

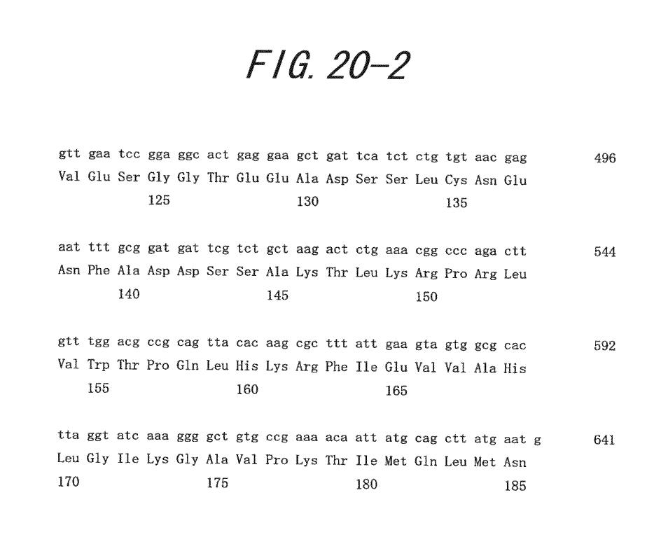

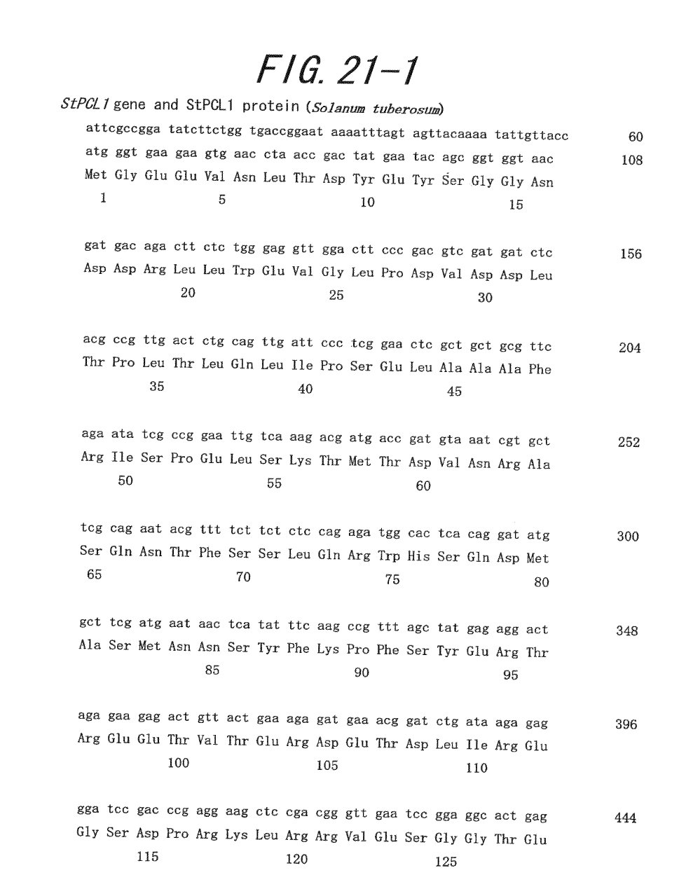

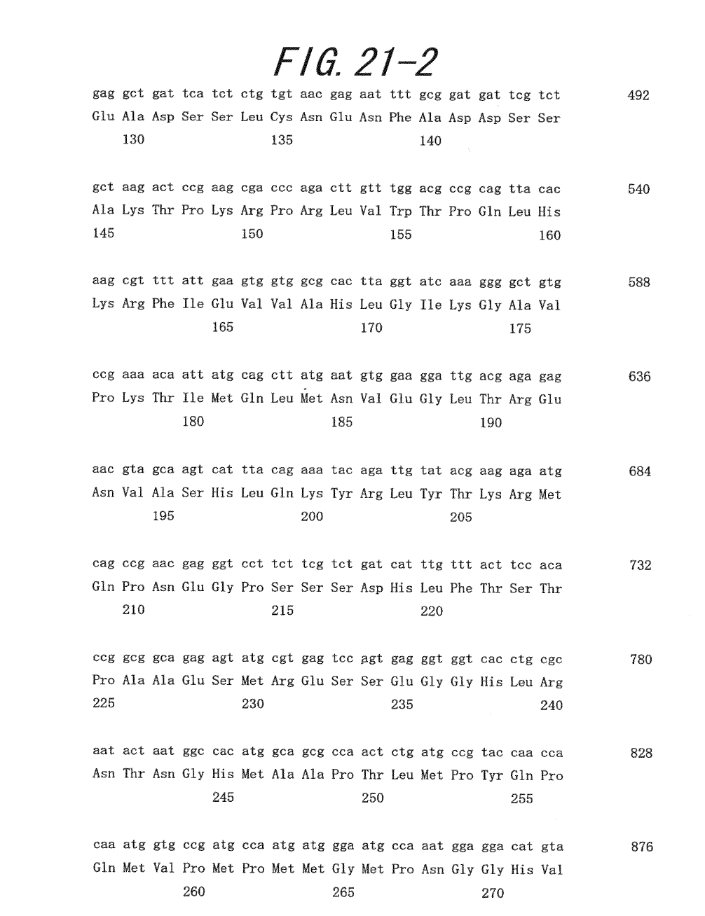

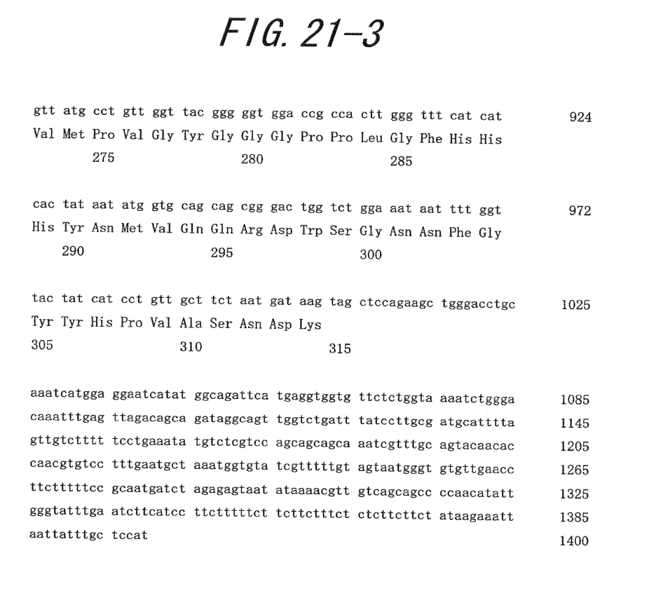

The model of the biological clock of plants based on this invention is shown in FIG. 14. The PCL1 gene forms a negative self-feedback loop, and this feedback loop is believed to be the essence of the biological clock. Also, the PCL1 gene controls the expression of the known clock-related genes TOC1, GI and ELF4 promotionally and controls the expression of CCA1 and LHY suppressively, and it is believed that such gene expression networks stabilize the circadian oscillation.

### Example 14

### Construction and Bioluminescence Rhythms of the PCLL::LUC⁺ Bioluminescence Reporter Strain

In order to monitor in real time in detail the expression of the PCL1-similar gene PCLL as bioluminescence, the PCLL::LUC⁺ bioluminescence reporter strain was constructed in the following procedure. First, a bioluminescence reporter gene cassette (PCLL::LUC⁺) connecting the promoter region of the PCLL gene to the coding region of modified firefly luciferase gene was constructed, and this cassette was inserted into the cloning site of binary vector pBIB-Hyg (Becker, Nucleic Acids Res. 18: 203 (1990)) to construct pBIB/PCLL::LUC⁺. According to the method of Clough and Bent (Clough and Bent, Plant J. 16: 735-743 (1998)), the T-DNA region of pBIB/PCLL::LUC⁺ (which contains hygromycin B-resistant gene HPT and PCLL::LUC⁺) was transferred via agrobacteria into the genome of Arabidopsis wild-type strain Col-0. The transformants obtained by gene transfer was selected as hygromycin-B resistant plants according to the description of Weigel and Glazebrook (Weigel and Glazebrook, ARABIDOPSIS: A Laboratory Manual Cold Spring Harbor Laboratory Press, Cold Spring Harbor (2002)), and then homozygous plants (T₃) having T-DNA inserted into a single genetic locus was selected and used as wild-type PCLL::LUC⁺ bioluminescence reporter strain.

The measurement of bioluminescence of the PCLL:LUC⁺ luminescence reporter strain was performed according to the method of the inventors (Onai et al., Plant J., 41: 1-11 (2004)). After resetting the biological clock by giving it a light/dark cycle of 12-hour light period/12-hour dark period or a cycle of 12-hour dark/12-hour light, the bioluminescence was measured under constant light or constant dark.

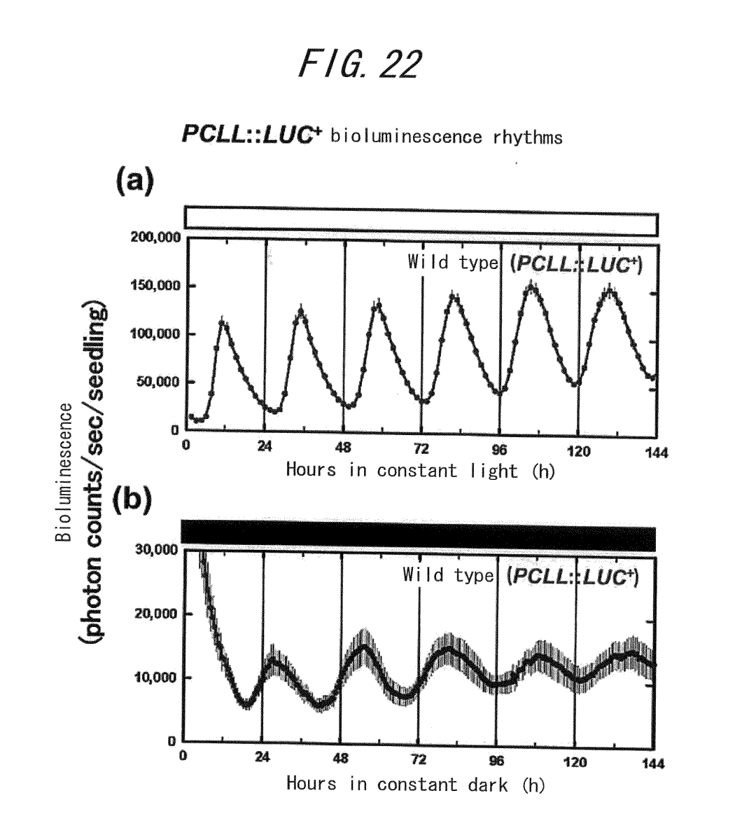

The bioluminescence pattern of the PCLL::LUC⁺ luminescence reporter strain is shown in FIG. 22. In the cases of both constant light and constant dark, the bioluminescence of wild-type PCLL::LUC⁺ luminescence reporter strain showed clear circadian rhythms matching the bioluminescence rhythms of the PCL1::LUC⁺ luminescence reporter strain. These results mean that the expression of the PCLL gene has the completely same pattern as that of the PCL1 gene.

### Example 15

### Construction of PCLL-Overexpressing PCLL-ox Plants and the GI::LUC⁺ Bioluminescence Pattern of PCLL-ox Plants

As shown in Example 11, when the PCL1 gene is overexpressed, circadian rhythms are disrupted. Assuming that the PCLL gene performs identical or extremely similar functions to the PCL1 gene, that is, serves as the clock gene, the overexpression of the PCLL gene is predicted to have a serious effect on circadian rhythms. Therefore, it was investigated how the circadian rhythms are influenced when the PCLL overexpressing plants PCLL-ox is constructed to disrupt the circadian expression of the PCLL gene. The PCLL overexpressing plants PCLL-ox was constructed in the following procedure. First, the PCLL overexpression cassette (CaMV35S::PCLL) connecting the 35S promoter of cauliflower mosaic virus (CaMV35S; base sequence of 4,951 to 5,815 registered at registration number AF485783 in the GenBank/EMBL/DDBJ database) to the coding region of the PCLL gene was constructed, and this cassette was inserted into the cloning site of binary vector pBIB-Hyg (Becker, Nucleic Acids Res. 18: 203 (1990)) to construct pBIB/35S::PCLL. According to the method of Clough and Bent (Clough and Bent, Plant J. 16: 735-743 (1998)), the T-DNA region of pBIB/35S::PCLL (which contains hygromycin B-resistant gene HPT and 35S::PCLL) was transferred via agrobacteria into the genome of Arabidopsis wild-type strain G-38 (the wild-type strain that has the GI::LUC⁺ luminescence reporter gene). The transformants obtained by gene transfer was selected as hygromycin-B resistant plants according to the description of Weigel and Glazebrook (Weigel and Glazebrook, ARABIDOPSIS: A Laboratory Manual Cold Spring Harbor Laboratory Press, Cold Spring Harbor (2002)), and then used as the PCLL overexpressing plants PCLL-ox.

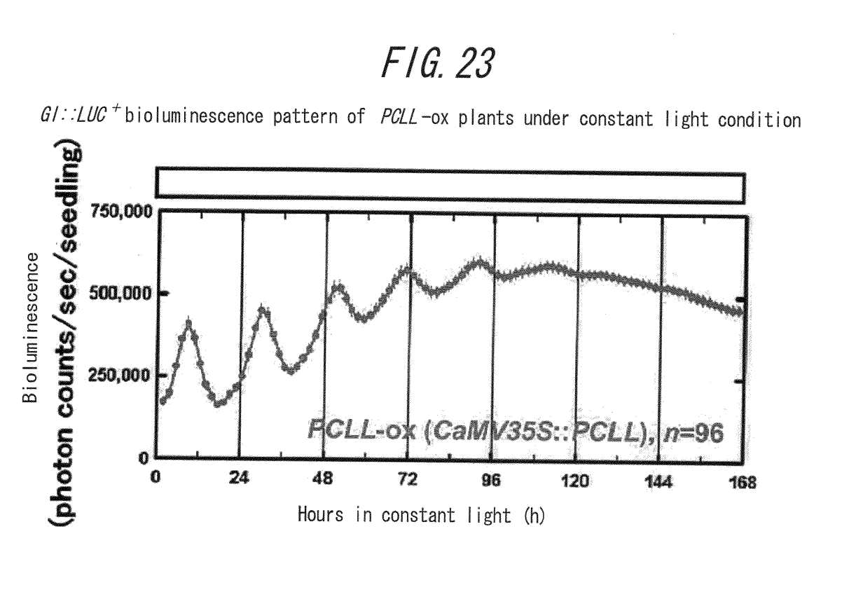

The GI::LUC⁺ bioluminescence pattern of the PCLL-ox plants under constant light is shown in FIG. 23. The measurement of GI::LUC⁺ bioluminescence was performed according to the method of the inventors (Onai et al., Plant J., 41: 1-11 (2004)). After resetting the biological clock by giving them a light/dark cycle of 12-hour light period/12-hour dark period, bioluminescence was measured under constant light. As a result, the bioluminescence rhythms of the PCLL-ox plants showed a cycle of about 2 hours shorter than wild-type while decaying gradually until the 5^{th} day, and disappeared thereafter. This result means that the biological clock fails to function correctly when the circadian expression of the PCLL gene is disrupted.

From the results of Examples 14 and 15, it was demonstrated that the PCLL gene have a similar base sequence to the PCL1 gene, as well as has an extremely similar expression pattern and functions. Therefore, it can be said that the PCLL gene is an important gene for clock function. Also, from these results, it is strongly suggested that PCL1-similar genes except for the PCLL gene described in this specification and PCL1-similar genes that are expected to be found hereafter have functions important for clock function.

1. A nucleic acid involved in the control of the biological clock comprising following (a) or (b):
(a) a nucleic acid comprising a base sequence represented by base sequence numbers 1-2846 shown in SEQ ID No. 1 of the sequence listing, or
(b) a nucleic acid wherein a part of said base sequence represented by the base sequence numbers 1-2846 is deleted, substituted or added, and having 80% homology with said base sequence.

2. A nucleic acid involved in the control of the biological clock comprising following (a) or (b):
(a) a nucleic acid comprising a base sequence represented by base sequence numbers 1-4554 shown in SEQ ID No. 2 of the sequence listing, or
(b) a nucleic acid wherein a part of said base sequence represented by the base sequence numbers 1-4554 is deleted, substituted or added, and having 80% homology with said base sequence.

3. A nucleic acid involved in the control of the biological clock comprising following (a) or (b):
(a) a nucleic acid comprising a base sequence represented by base sequence numbers 1-4700 shown in SEQ ID No. 3 of the sequence listing, or
(b) a nucleic acid wherein a part of said base sequence represented by the base sequence numbers 1-4700 is deleted, substituted or added, and having 80% homology with said base sequence.

4. A nucleic acid involved in the control of the biological clock comprising following (a) or (b):
(a) a nucleic acid comprising a base sequence represented by base sequence numbers 1-1505 shown in SEQ ID No. 4 of the sequence listing, or
(b) a nucleic acid wherein a part of said base sequence represented by the base sequence numbers 1-1505 is deleted, substituted or added, and having 80% homology with said base sequence.

5. A nucleic acid involved in the control of the biological clock comprising following (a) or (b):
(a) a nucleic acid comprising a base sequence represented by base sequence numbers 1-400 shown in SEQ ID No. 5 of the sequence listing, or
(b) a nucleic acid wherein a part of said base sequence represented by the base sequence numbers 1-400 is deleted, substituted or added, and having 80% homology with said base sequence.

6. A nucleic acid involved in the control of the biological clock comprising following (a) or (b):
(a) a nucleic acid comprising a base sequence represented by base sequence numbers 1-641 shown in SEQ ID No. 6 of the sequence listing, or
(b) a nucleic acid wherein a part of said base sequence represented by the base sequence numbers 1-641 is deleted, substituted or added, and having 80% homology with said base sequence.

7. A nucleic acid involved in the control of the biological clock comprising following (a) or (b):
(a) a nucleic acid comprising a base sequence represented by base sequence numbers 1-1400 shown in SEQ ID No. 7 of the sequence listing, or
(b) a nucleic acid wherein a part of said base sequence represented by the base sequence numbers 1-1400 is deleted, substituted or added, and having 80% homology with said base sequence.

8. A probe comprising the nucleic acid described in claim 1.

9. A method comprising using the probe according to claim 8 for screening a gene involved in the control of the biological clock in living organisms.

10. A peptide fragment involved in the control of the biological clock comprising following (a) or (b):
(a) a peptide fragment comprising an amino acid sequence represented by amino acid sequence numbers 1-323 shown in SEQ ID No. 8 of the sequence listing, or
(b) a peptide fragment wherein a part of said amino acid sequence shown in the SEQ ID No. 8 is deleted, substituted or added, and having 80% homology with said amino acid sequence.

11. A peptide fragment involved in the control of the biological clock comprising following (a) or (b):
(a) a peptide fragment comprising an amino acid sequence represented by amino acid sequence numbers 1-298 shown in SEQ ID No. 9 of the sequence listing, or
(b) a peptide fragment wherein a part of said amino acid sequence shown in the SEQ ID No. 9 is deleted, substituted or added, and having 80% homology with said amino acid sequence.

12. A peptide fragment involved in the control of the biological clock comprising following (a) or (b):
(a) a peptide fragment comprising an amino acid sequence represented by amino acid sequence numbers 1-238 shown in SEQ ID No. 10 of the sequence listing, or
(b) a peptide fragment wherein a part of the said amino acid sequence shown in the SEQ ID No. 10 is deleted, substituted or added, and having 80% homology with said amino acid sequence.

13. A peptide fragment involved in the control of the biological clock comprising following (a) or (b):
(a) a peptide fragment comprising an amino acid sequence represented by amino acid sequence numbers 1-312 shown in SEQ ID No. 11 of the sequence listing, or
(b) a peptide fragment wherein a part of said amino acid sequence shown in the SEQ ID No. 11 is deleted, substituted or added, and having 80% homology with said amino acid sequence.

14. A peptide fragment involved in the control of the biological clock comprising following (a) or (b):
(a) a peptide fragment comprising an amino acid sequence represented by amino acid sequence numbers 1-70 shown in SEQ ID No. 12 of the sequence listing, or
(b) a peptide fragment wherein a part of said amino acid sequence shown in the SEQ ID No. 12 is deleted, substituted or added, and having 80% homology with said amino acid sequence.

15. A peptide fragment involved in the control of the biological clock comprising following (a) or (b):
(a) a peptide fragment comprising an amino acid sequence represented by amino acid sequence numbers 1-185 shown in SEQ ID No. 13 of the sequence listing, or
(b) a peptide fragment wherein a part of said amino acid sequence shown in the SEQ ID No. 13 is deleted, substituted or added, and having 80% homology with said amino acid sequence.

16. A peptide fragment involved in the control of the biological clock comprising following (a) or (b):
(a) a peptide fragment comprising an amino acid sequence represented by amino acid sequence numbers 1-314 shown in SEQ ID No. 14 of the sequence listing, or
(b) A peptide fragment wherein a part of said amino acid sequence shown in the SEQ ID No. 14 is deleted, substituted or added, and having 80% homology with said amino acid sequence.

17. A peptide fragment involved in the control of the biological clock comprising following (a) or (b):
(a) a peptide fragment comprising an amino acid sequence represented by amino acid sequence numbers 1-121 shown in SEQ ID No. 15 of the sequence listing, or
(b) a peptide fragment wherein a part of said amino acid sequence shown in the SEQ ID No. 15 is deleted, substituted or added, and having 80% homology with said amino acid sequence.

18. A peptide fragment involved in the control of the biological clock comprising following (a) or (b):
(a) a peptide fragment comprising an amino acid sequence represented by amino acid sequence numbers 1-200 shown in SEQ ID No. 16 of the sequence listing, or
(b) a peptide fragment wherein a part of said amino acid sequence shown in the SEQ ID No. 16 is deleted, substituted or added, and having 80% homology with said amino acid sequence.

19. A probe comprising the peptide fragment described in claim 10.

20. A method for screening a gene involved in the control of the biological clock comprising conducting said screening with the probe described claim 8.

21. The method according to claim 20 wherein the screening is conducted by using at least one method selected from the group consisting of situ hybridization method, Southern hybridization method, determination of total base sequence, colony hybridization method, plaque hybridization method, Northern hybridization method and Southwestern method.

22. The peptide fragment according to claim 10, wherein said peptide fragment controls the transcription of certain genes in the nucleus of cells, and oscillation and stabilization of circadian rhythms.

23. A peptide fragment according to claim 10, wherein said peptide fragment has a DNA binding motif belonging to the GARP family.

24. A composition for controlling the biological clock of plants comprising the peptide fragment described in claim 10.

25. A vector comprising DNA or RNA according to claim 1.

26. A transformant holding DNA or RNA according to claim 1 in a manner that enables its expression.

27. A method for producing a peptide fragment involved in the control of the biological clock comprising following (a) or (b):
(a) a peptide fragment comprising an amino acid sequence represented by amino acid sequence numbers 1-323 shown in SEQ ID No. 8 of the sequence listing, or
(b) a peptide fragment wherein a part of said amino acid sequence shown in the SEQ ID No. 8 is deleted, substituted or added, and having 80% homology with said amino acid sequence
comprising a step of culturing the transformant described in claim 26.

28. A peptide fragment involved in the control of the biological clock comprising following (a) or (b):
(a) a peptide fragment comprising an amino acid sequence represented by amino acid sequence numbers 1-210 shown in SEQ ID No. 8 of the sequence listing, or
(b) a peptide fragment wherein a part of said amino acid sequence represented by amino acid sequence numbers 1-210 shown in the SEQ ID No. 8 is deleted, substituted or added, and having 80% homology with said amino acid sequence.

29. A peptide fragment involved in the control of the biological clock comprising following (a) or (b):
(a) a peptide fragment comprising an amino acid sequence represented by amino acid sequence numbers 1-143 shown in SEQ ID No. 8 of the sequence listing, or
(b) a peptide fragment wherein a part of said amino acid sequence represented by amino acid sequence numbers 1-143 shown in the SEQ ID No. 8 is deleted, substituted or added, and having 80% homology with said amino acid sequence.

30. A probe comprising the peptide fragment described in claim 28.

31. A method for screening a peptide fragment involved in the control of the biological clock using the probe described in claim 30.


SEQUENCE LISTING (16 sequences)

SEQ ID NO: 1 - DNA, 2846 bp, Arabidopsis thaliana
cacaacaaacgccgacggtgactgagaattccaccagctacgataccaacttgatgacgatggttgagaaatccgttgcggagaagcggaagcgttctgagaagaagtagcgaggtcatcgaccacaatcaaggagtaatatagacaataataacgaataaaaccctaatctgtactaatcactcgaagaagatgaaaaattggggaaatctcagagaatcagaaatttgagaagaagataacgtttcgtcagtttgtgaagttggggcatcgaacccgtgacgttgaagaagctaaacagagtaactaaacctctctgagcctttccacaataattattttcattttcttaaaagttaggtgccagccaatctgtgccgttagattgatctttatatgcttctataaatatctaaaacaacaatccaacggtggaaagatcacattgcgtgtcagtactgtagatactcatcaccgaatctttctcctcttttcactctccagctggccgaacccaccggttttgcccactttctctctaggcccgcaaacacaacttgctaagtcggtagtaatttactgaaaaaccctcacaaatagctgacttggctcttttcactccacgtggctccatcttacgtgcctagtttggtaatttcaatttcaatcacctagatcgtaactattgatacataatgtgaactgatgacgtgttgaaaagagattggttaggtcggtttgaaaagatatttagaagatctaaaatatctagctaactcgtccacgttggaattaaaatattgcgctttttcctggaaaagaaaaatataaaacaagtgataatcttctctttttcgaaattgtttcacttctgaaaaaaaaaaaaaaaaaaattgtttgctcatcatcttcacaaaccaaacttctcttcttctccgatctcagatttgaatttttatcgattttgagcttcgaagagctcaatctctaactgaatttcgagaaattagagagtttgaaatgggagaggaagtacaaatgagcgattacgatgtttccggcgacggagatagggtttctgaatgggagatgggattaccaagcgacgaagatctagcatctctttcttactctctgattccaccgaatttggcgatggcgtttagtatcacaccggagagaagccgtacgattcaggatgtgaatcgtgcatcggagacgacgctctcgtcgctacgtggtggatcttcaggtccaaatacctcgtcgtcgaataataacgtggaggaggaagatcgagttggatctagcagtcctggatcggattcgaagaagcaaaagacatcaaacggtgatggagatgacggtggcggtgtggatccggattcggcgatggcggcggaagaaggagattcaggaactgaagatctatctgggaaaacacttaaacgaccgcgtttagtgtggacaccgcagctacacaagagattcgttgacgttgtagctcacttagggatcaaaaacgctgttccgaagacgattatgcagctgatgaacgttgaaggattaactcgtgagaacgttgcgtctcatcttcaaaagtataggctttacctcaaaaggatgcagggattgactaatgaaggtccctctgcttcggataagctcttctcttcaacacctgttcctccacagagcttccaagatatcggtggcggtggcggtagcagcggtaatgttggagtgccgattccgggggcgtatggaacgcaacagatgatgcagatgccagtttatgcacatcatatgggtatgcaaggatatcatcatcaaaatcataatcatgatccttatcatcagaatcatcgtcatcatcatggagctggtggaaatggtgcgtttgagtcaaatccttatatgatgcagcagaataagtttggatccatggcttcttatccttctgttggaggtggaagcgcaaatgagaattaaaaagagtctatagatggattcaatgggggaggaagagataggtgagcttgttattgctcgtggaactgataattattgtcaagtttctgtttttttaagtacagtacagttataattcttactcgatttgatcgaaattttcatcaagtttctgtcttttactataaatggtatacaataaagtcttaataatcacttgtttggtgatgttgacagagttaatataatcaactaagttgaatttgattttgagattcttgtgctatgttgaaatgttattgagaattaagtatcaaaattgctctcatgtgacttgagacttaatgtaatccagatcatgtttgattcatcttcggaattatatggtggttttgaaaaccatgtgtttaattttctcttcgggactaggtaactaacactgtgagttatgtcaaatccaagagagcatatgtgtcatgtggctcgacattttcttgtgtactctttttgcagaagtgatagtgtcatgttattttggttttggtttcctcattgggaaagtcttgtgatgttttttgatctagacaagatattacctgtgcaagcttttaggtagaatgagcagtttctcttttatgtttgggttctgcatcttttgcaggtcttgatcatatttgattgttgttacaggttcttggtgtctacaaaatttcttatcttttgcggagttttccaccgcaaagtcccaaattgcatctgttcaggagaggatcatacaaagtatcatgtagaagctaaaccaagagaagactgcagacaaatttcatatactcatttttctatcttgtaagaggtaagtccctttgttttatttg

SEQ ID NO: 2 - DNA, 4554 bp, Arabidopsis thaliana
ctttgcttaactctgatctatctactttgagattgacaaaaatcttcgataccgagcaaaattaaacttacgattagagacgaaaacacaggaggaagatcgaagaagcttgcttgaaatacagaggaagcaaagaacgcttcgaatatgtggtgaccgagtggactcagtgagacatttgtgagtttggtgcgccgagtcgcgcggcgataaagtgaatcagatgatttgtacgcgaagatttggtaaacaggtattgggctagaggaaatggaatgggtctagatacggcccaataaaggcgcgagatagagacaactgaagtcttgtttcctgttatttcttttgattttgttttactaatctaaatatactaaatcagcagtcaattttccaataactgatgcattcagtgattgacacatcagcttagtactacttttttattttttcatttttacgcatagtatgtgtcgtttttatttaactttgtttcattctcaaacgattttctctttcatccccacgatctccacatctccactttctctcttccaaaaactccatgactactgtgtaaaatctcaaagatgggtttctcaacatcattaatcgattactcagaaatcaggtactttgttcatctcaatgaaccaatttaccatcttctatttttgtttgtaattttgttttatatatatagaattctccatagatccgaaaatcatatatccttcttctagatttctatggcttttttttttttttgctttgtttctgctttatttgactgaattcgatttctctcatacatctccatttttccataggattattctttaatacaatcaatgcctatgattctcccgtctcacaaacactcaaagaacaacgacaaattttggcttctcatctgaacagttgccttaaaacaagggtgagtttaatatcttaatcgaagctcaagtgtttttttaatggtaaattaattctcatactttttactattcttctttcatctcttattttacttctttatatttgcaatttcttttttgtattaaaccgcaattgtattttcacgcaagatttttgtattaatacaaatacttcaattttaaagtcatctcttattttacttctttatatctgcaatatctttgatattgtttcaaaggcagcgtctgcgttatgtagttacttacttgtgcacattttacatttttttttagacttccaagctttgggtgtcaacgaatgtttgtaaactgaattactgatgatgagaagaagaacacattacaattggttggagctaatgatgaaataagcggacatgtatatacccagcataattttcgtacttatactctcttatatttctaaactcttgatttgttagttactgtctatcgcaattgattcattgggtagaaaagttgtaccttctttatatctttttcttaatctttgtatattcttcagtcaatgataataaatttgttgtatatttatgctgtctgctttattttaaaaatcttagttaagagttgtagcagatataatttaaaacaatgttgtccctgcatagtaggggccaaatgctagtagtagtttaaaagtaaagtacgtaaaataaaataacgtgtatttttcattataggaaatgtagtttaggccgttattgttctttttcattataaaataacgtgtattttatgtgttgctgtttaaccctaaactcagacagcgaatgttgagttccgatgcagtggtgtatggtttgcggatacagtaaaacttttataaattaataatattagaattgcgaaattttgttaatttaaacaaatattaatttatagagaaattttttaaatttaataattgttttaaaaattgaactcttttcgtagcccaaacccaccggtattgaactctttttggcccacttagccacgttaaagggtcacttgttactagttgtaattgcaactatctgcctaaaccgtcgtgccttttgtctacattctagaaccatgcactaatgacgtggctggagataagttggttgattacacacgtgtcacttagacagagcgagtagactttgatgcgggtaaaagatattttagattatctagctcgctaggccacgttggcggatatttgcactttttctttttttaaaaagaaaaaaaactaggctctacgaattttccggtcaccttctccgatctctgatccgatatttttccacatgttgttgagctttaaaggatttgaaaccgttatgaaagttatgaaaacgaagagtgtgtgaaacatggggaaggaagttatggtgagcgattacggtgacgacgacggagaagacgccggcggcggcgatgaatataggattccggaatgggaaattggtttacccaacggagatgatttgactccgttatctcaatatctagtcccgtcgattctcgcgttagctttcagcatgatcccagaacgaagccgtacaattcacgacgtcaatcgcgcgtcgcaaatcacgctctcttcgttgagaagcagtaccaatgcttcgtctgtgatggaggaggtcgtggatcgagttgaatcgagtgttccaggatcagatccgaagaaacagaagaaatcggatggtggtgaagcagcggcggtggaggattccacggcggaggaaggagactccgggcctgaagacgcgtctgggaagacatcgaaacgaccgcgtttagtgtggacaccgcagctacacaagagatttgtggacgttgtggctcatctagggattaaaaacgcagtgccgaagacgattatgcagctgatgaacgtggaaggacttactcgtgagaacgttgcgtctcatttgcagaaatataggctttaccttaaacggattcaaggattgacgacggaagaagatccttattcgtcgtcggatcagctcttctcttcaacgccggttcctccacagagctttcaagacggcggaggaagtaacggaaagttgggggttccggttccggttccgtcgatggtgcctattccaggctatgggaatcaaatgggtatgcaaggatattatcaacagtatagtaaccatggcaatgaatcaaaccaatatatgatgcagcagaataagtttggaacaatggtgacatatccttctgttggtggtggtgacgtgaatgacaagtaaatggatcttaaaggtctataatttgctctacagagagatactggtgagcttgttttcgtcttcttctttgagattcttattctttgatgtttgttcatgttactagcaaagtgttacaattttcttggtagagtgttatggtcaaatcctagtttggtgttgttatatatttactctgtctttgcaagtagttgatcttaattgattcttgttacaggttcttggcttatggtttattttcccacttcatgaggttgttgtgacttttaattctccatgttttccacacaagtctttattgcctttgtatagaaaatgatttcgagaaaatcactgggaagcttggtattgttggaggatgaagccttctatgaatgatttagtttcctactgtctccattctttatgaggtaataaagccttcttttgctcatcgcttgtagtcttcttaaattcaagacagcgtcacatgtttgttcggttatgttaattgtttctttctttggataatgaagatagcatcaggtctcatgtctcctcactttgataaatatctctttcatttatcattttcttctattaaaatagtttttctctttcttaagaaaagtatcttttgattgtgttggatggtcatacaccaattaactcccaaccacaaggaagaaagtgacatttcaaccataagctcgtgtttctttgcccacaatgagttagtagttttagccttttagccatatagttttcatttggtaaataatctgttaagatctttaaatctgcaaactagatgagaatccatagaatatttctgtgaccaatgtagagtcgccattagaagatcacttttcacctctctgttttgtttcattcaaaagatgtttctctaacgttatcttcatattacttctttgtgggtcactcgtgtctcctctacccttcctaaacttacaacaactgactcaaactgtccttgaaatttccaatgcgccagtcttttcatgggacttagttgagctgtttagtttggtgacagtgaaatattattaaagtaacctaaatgaaagtttcaagtggttgaaacgtggcctcagcaactgtaatggagaagcccaagctctttatctcttatcatgtgaacaatgattttgtctataaatgcagaaactcttttatttttagatacaactgccaaaacattactctaaaaatagaaaccatctccttttatgccatttgattcatgatgattataaagaatctcttctgatacagttttgacttcaaaacagggactacatgaaattcatcatttttaaggaattgacaaa

SEQ ID NO: 3 - DNA, 4700 bp, Oryzae sativa
tttgtgtgactaggatatgatgatttgttatatataacacaaagctgtttttgtgtgtgcgcagaaaagcggcttgggcttcaaccatcaactgcagcttgaagaataagcgctatctagttgtacagctaacagtggctaggtttgcactggcgtcatgtttttctatctaaaccaacccctactagtacgaggctctgttctgaatggccttgttagaaataaagagagagagtagtagcttgcaccggagagtgtacattctgtagctgaggaactagcacaagggagggagataaaaagaatcttcatgtgtaccgtgtactatgagttctcaaactgtgtaaaaagaggaggagcaaggtgtacaaattttttagaactagagcgaaagagagggctgttgttagctgttttattaaacttttaaaaagccagtcactagtacttttttctttttgaaaaaaaaagaattaatctcagccctgcccggtatcgatgattcgatatcatatcagggcgtcgcgttttgttgtaacatatactactactactgtaccacagatgatgccttgagctgttctgtgggtgaagctggagctattaatctcagctttgctcttttttactactacatttatcatctactggtcgcttgcttgttcttttcagggtagatcgtgtatcattatcgtcatctttcttgtttctggacatattattataatctaaatgcagcaatttataacagtactaaggtggtgttctttttctcctgaagatgaagattaagtttttttacgcaaaacgatgtggtattaacttatgattgattgtgttttaattattacaaacttgaaaaataaattaatatgctattttagggcaacttttatatagaaagttttcgcacgaaacgcaccgtttaacagtttaaaaaacatgccacgagaattcaaaattccatccattcttttcattagaaacgaatgggacctaataaaacactacatcctaaaatatacagtaacagtataacatttgataggatacattcgtactagatagatgaagtctatatttcatttgattcgaaattgctagtatattatttatgggatatgggtgttactactaatgtaatttttatttggaacataggcagtataaaagaaggatagactgtatagactaaggttaggagagtttattttggttgctgaagactgaaggggagtagggtgggagaagagaagagaaatatctggttgctccagcttggcatcgttgaccgggccatcccaacccaatccaatccacagaatcgcatcgcatcgcccacaattttcaattattatacaggaagaagaaaagggaaaggcggagagcagagccacgccgtcggattcgccctatccgaatcttgaggaaatatctccagtcctaatcagcttgaggagtgccccgaaaagagcacgggaataacgatcgtgccactgcaaaagggcgcgggcggacggccgggatgcaaaagcgcatcccccgaatcaaaaatatccggctcaccggtccacgtggaccgtgatctccctttgctctcttctctccgtccccacccacctccactttgctctttattccccttttcccctctttttttattactatttattcgtcttccccctcttgtcttccacctcgactcaactcaactcgcacaaatatcttctctcttctcacccaccgcccgattcttcgagtccccgatttggttcggcctgaagaagggtctgggtctctctagagagtgagtagccatcgatttgagatttgattagtaattcggaggaggaggaacgagtggtttttttgatttgatttgatttgattgagaagaaaatccgagtattatgggcgaggaggcgccggaggagtacgagctgggcggcggggaggacgagcgggtgatggagtgggagacggggctgcccggcgccgacgagctgaccccgctgtcgcagccgctggtgccggcggggctggcggcggcgttccgcatcccgccggagcccgggcgcacgctgctcgacgtgcaccgcgcgtcggcggcgacggtgtcccggttgcggcgcgcgtcgtcgtcgtcgtcgagctcgttcccggcgttcgcgtcgaagggagcgggaacgggagcggacgaggcggagtcagggggaggcgcggatggggggaacgggaacaccaacaacagcagcagcaagagggcgcggctggtgtggacgccgcagctgcacaagaggttcgtggaggtggtggcgcacctggggatgaagaacgcggtgcccaagacgatcatgcagctgatgaacgtggagggcctcacccgggagaacgtcgccagccacctccagaagtatcgcctctacgtgaagcggatgcagggcctctccaacgagggcccttccccctccgaccacatcttcgcctccacccccgtcccccacgcctccctccacgaccaggttccttctccttaccacccccacccccaccaccactcctacaacaacgccgcctatgccgccaccgtctcctcctaccaccactaccaccacgccaaccactgatccattccttctcatcatcatttcatttccacatctatataccacatactactccataacgaaggtgtttcatcaataagagaagagaagagaagagaatgctccctcaactgtaaactcctaattctaattaagggcaaagtgtatccatccatccatccacgctgtttgtttgtagctgttgttaattgagctggatgcatgacatgtatctctatgtacctacctgcgtgacaaaggcatcatgcatatgtatggttcccttgcaatttggggaatccagaaatgattcatacttgtgattcttgcaattgtgatgctcacactgtactctacacattcagttccataaaaaacaaaactaatactacgaatatagatgcgatatatactattactacaaatctatctagatggtacacatacttttctctttgtgtgatggcaaatgaactgaatagcgaataaatacatatccattcatcatagataatactaataaaaccctatcacatcggatgtttggacactaattataaatattaaacatagactattaataaaacccatccataatcttgtactaattcgtgagacgaatctattgagcctaattaatccatgattagcctatgtgatgctacaataaacattatctaattatagattaattaggcttaaaaattttgtctcgcaaattaactttcatttatgtaattagttttgtaagtagtctatatttaatactctaaattagtgtctaaatatagagactaaagttaaatctatggattcaaacaccacctaatatggtactaggttgtggaatagcaaggacagagagatttgttctagatagatattgggtacacatgacaagattgattgctgcaaggaagagcaagtggctagactacttatttgatgaagaaatgtaatgtgtggagcagcggagaacagcactgatggtgacgagtcccatgaggcatgaggagcggaggagacaagtgcacgtcgcctgcctctctgtctgtatctctactatgtatgtaggagtaaatgtatcagtatcacggtcgtttttatcctttccatgccatgcctgagagaaatgaattggcttcggcttcggattggatatgcttcttcctccaccacacaaccagagcacaagccaaggcaaacctcactgttgcttcccttcccttcgccctgtaccaatagaagttcggattgggtttcgttcttttcttcttcttttttttttcccttgtaccattagaacatggccatgccacatattgccatcgctccatcgctaacatttccacgacggccggcagcctctctcctattaccaaatttcaaaagcttctatacaaactagtgtcaaactagtgtagtactctattcgttttttaatatatatggtactgttgattttttaagatatatttaattattcatcttattaaaaaaataatgtatttatttattttattatgacttgatttatcgtcaaatgctcttaaaattttttatatttgcacaaaaaattttaaataaaataaatgattaaatatttataaaaaaatcaactgcatcatcatgtatttaaaaatcaactgcctcccaacaacatctatcaccggtgaaagcaagaaaaatagttagttcagggatatcatatgctgcctgtaatgtattggatccatctgtactccattatcacgcacgcctctccaagaaatcataagctcattagcggtcaacacgatgcgataccgccataatcgactacagtctacaaatttaggccaatttcaaaatttgtgtggagtataaattacaacgaattttctgcatttgctcttgtttgttcgtacggatagatactactagtccgtcaattgccaataggcagggagaaaggaaaaaaaaaacaaaaaaggaaaaaagagctttgagttgactgacatggccgtgctctcctcttcctcccatggcctcctccacccaccgctccgtctcctcgccgccgcctccgccagtagcggcagcagctactcgatt

SEQ ID NO: 4 - DNA, 1505 bp, Nicotiana benthamina
gatcccaccccacactctccgtcttcttctcgccggtgaaatggacaggaaaatcctctctttctctgatccaatcctctactgaaatcacggcggatttcattaacttgatttttttaaattgtaataaaaaaaattgtaagaagttttatgggcgaagaagtaaaaatcaccggcggtgaggcgttcgccggagacgataaccgtattctcgagtgggaggacggacttccgagtctcgacgatataacgccgttatctcaggcgttaatcccaccggaactcgcgtcggcgttcaaaatatcgccggagccggcgaagacgatgaccgacgtcaatcgtgcttcggagaacacgttctcatctctccgaggcagtggaccgttgcaacaattacagattctgtcttcaccggaaacggaacacggatcggatccgaggaagacccgaaggattgacccggagatgttagaagcggattcagcattaccgaggaatgagaattgtggaggaggagatgatagtaataataagtcagcttctaagacgctgaacaagcggccacggctcgtgtggacgccgcagctgcacaagagattcgtggacgtggtggctcatttagggataaaaaatgcggtgccaaagacgattatgcagttaatgaacgtggaaggattaacgcgggaaaatgttgcaagtcatttgcagaagtataggttgtacttgaagagaatgcaagggctgtcaaatgaggggcccacttcttccgatcatttattcgcgttcacacccgtgccacagagtctgcagcagtcaggtggtaatggccagaatggtaatggccataacagtagcggttataacagtaacggtcataatagtaatggtcatgcgcagatgccaatgcaaatgcctatgccaatgccaatgatttatcccccacaaatggtgccaatgcctatgatgggaatttcaggacacggacatggacatgggtttcatcatcagtataatatgggggcacaacagcgagactggtctggaaataaatttggttcttatcatcatgttgctcctagtgacgaatagctatagtagacaagaaaaaagagggttggttactggattggtccatcaagaagattctcacttggtggattgcaaactaggaattccagttagtatcatttaggctttttttacttgtttagatttggtggatttatagggacaaaaatttcataaattgctctaaatttcttaaagcatattggcctaagatgagtttaaacgagtaacatggttagtgaagattcatatagctaaccccaacttatttgggactaaggcaaactagtagtagttgctccaaattttttagagccatgtcatttcgtgttgtagcttcttattcagcaaggagcttaatacataattagagaacaagatagaactgttgtttcgatcacacttttcttgaaagatagaacaataatttcggattc

SEQ ID NO: 5 - DNA, 400 bp, Nicotiana tabacum
cttctcctccattttctttccttcaccaataagcctttgaaatccttcggcatatcttcatcaaatcgatatctgctgctctgtcccgccgaagatttaatttaacttgcattaacaggaattaaggaagaaccctaagagtgtgaattttgtccagttgaacaacttttctgtaaccagctagctaccatgggtgaaaaagtgaagctttctgagtacgattctagcggtggtaataatgatgacagagttttgtgggaggttggacttcccggcgccgacgatctcacgccgttgactctgcagttgattccggccgaacttgcttctgcgttcagaatctcgccggaatcctcaaaatccatgactgatgtaatcgcgcttctcagaatactttttc

SEQ ID NO: 6 - DNA, 641 bp, Lycopersicon esculentum
cttcattcaacggcatctctctcaaattcgccggaatatactccaccagcggaagaagattttagtacttgcaaatactgttaccatgggtgaagaagtgagcctgaccgactatgaatcaagcggtggtaacgatgacagacttctctgggaaattggacttcccgatgtcgatgatctcacaccgttgaatatgcagttgattccgtcggaacttgctgctgcgttcagaatttcgccggaattgtcaaagacgatgaccgatgtaaatcgtgcttcgcagaatacgttttcttctctccagagatggcactcacaggatatggcttcgatgaataactctaatttcaagacgtttagctatgagaggagtagagaagagactgttactgaaagagatgaaacggatctgataagagagggatccgactcgaggaagctccggcgggttgaatccggaggcactgaggaagctgattcatctctgtgtaacgagaattttgcggatgattcgtctgctaagactctgaaacggcccagacttgtttggacgccgcagttacacaagcgctttattgaagtagtggcgcacttaggtatcaaaggggctgtgccgaaaacaattatgcagcttatgaatg

SEQ ID NO: 7 - DNA, 1400 bp, Solanum tuberosum
attcgccggatatcttctggtgaccggaataaaatttagtagttacaaaatattgttaccatgggtgaagaagtgaacctaaccgactatgaatacagcggtggtaacgatgacagacttctctgggaggttggacttcccgacgtcgatgatctcacgccgttgactctgcagttgattccctcggaactcgctgctgcgttcagaatatcgccggaattgtcaaagacgatgaccgatgtaaatcgtgcttcgcagaatacgttttcttctctccagagatggcactcacaggatatggcttcgatgaataactcatatttcaagccgtttagctatgagaggactagagaagagactgttactgaaagagatgaaacggatctgataagagagggatccgacccgaggaagctccgacgggttgaatccggaggcactgaggaggctgattcatctctgtgtaacgagaattttgcggatgattcgtctgctaagactccgaagcgacccagacttgtttggacgccgcagttacacaagcgttttattgaagtggtggcgcacttaggtatcaaaggggctgtgccgaaaacaattatgcagcttatgaatgtggaaggattgacgagagagaacgtagcaagtcatttacagaaatacagattgtatacgaagagaatgcagccgaacgagggtccttcttcgtctgatcatttgtttacttccacaccggcggcagagagtatgcgtgagtccagtgagggtggtcacctgcgcaatactaatggccacatggcagcgccaactctgatgccgtaccaaccacaaatggtgccgatgccaatgatgggaatgccaaatggaggacatgtagttatgcctgttggttacgggggtggaccgccacttgggtttcatcatcactataatatggtgcagcagcgggactggtctggaaataattttggttactatcatcctgttgcttctaatgataagtagctccagaagctgggacctgcaaatcatggaggaatcatatggcagattcatgaggtggtgttctctggtaaaatctgggacaaatttgagttagacagcagataggcagttggtctgatttatccttgcgatgcattttagttgtctttttcctgaaatatgtctcgtccagcagcagcaaatcgtttgcagtacaacaccaacgtgtcctttgaatgctaaatggtgtatcgtttttgtagtaatgggtgtgttgaaccttctttttccgcaatgatctagagagtaatataaaacgttgtcagcagccccaacatattgggtatttgaatcttcatccttctttttcttcttctttctctcttcttctataagaaattaattatttgctccat

SEQ ID NO: 8 - protein, 323 aa, Arabidopsis thaliana
MGEEVQMSDYDVSGDGDRVSEWEMGLPSDEDLASLSYSLIPPNLAMAFSITPERSRTIQDVNRASETTLSSLRGGSSGPNTSSSNNNVEEEDRVGSSSPGSDSKKQKTSNGDGDDGGGVDPDSAMAAEEGDSGTEDLSGKTLKRPRLVWTPQLHKRFVDVVAHLGIKNAVPKTIMQLMNVEGLTRENVASHLQKYRLYLKRMQGLTNEGPSASDKLFSSTPVPPQSFQDIGGGGGSSGNVGVPIPGAYGTQQMMQMPVYAHHMGMQGYHHQNHNHDPYHQNHRHHHGAGGNGAFESNPYMMQQNKFGSMASYPSVGGGSANEN

SEQ ID NO: 9 - protein, 298 aa, Arabidopsis thaliana
MGKEVMVSDYGDDDGEDAGGGDEYRIPEWEIGLPNGDDLTPLSQYLVPSILALAFSMIPERSRTIHDVNRASQITLSSLRSSTNASSVMEEVVDRVESSVPGSDPKKQKKSDGGEAAAVEDSTAEEGDSGPEDASGKTSKRPRLVWTPQLHKRFVDVVAHLGIKNAVPKTIMQLMNVEGLTRENVASHLQKYRLYLKRIQGLTTEEDPYSSSDQLFSSTPVPPQSFQDGGGSNGKLGVPVPVPSMVPIPGYGNQMGMQGYYQQYSNHGNESNQYMMQQNKFGTMVTYPSVGGGDVNDK

SEQ ID NO: 10 - protein, 238 aa, Oryzae sativa
MGEEAPEEYELGGGEDERVMEWETGLPGADELTPLSQPLVPAGLAAAFRIPPEPGRTLLDVHRASAATVSRLRRASSSSSSSFPAFASKGAGTGADEAESGGGADGGNGNTNNSSSKRARLVWTPQLHKRFVEVVAHLGMKNAVPKTIMQLMNVEGLTRENVASHLQKYRLYVKRMQGLSNEGPSPSDHIFASTPVPHASLHDQVPSPYHPHPHHHSYNNAAYAATVSSYHHYHHANH

SEQ ID NO: 11 - protein, 312 aa, Nicotiana benthamina
MGEEVKITGGEAFAGDDNRILEWEDGLPSLDDITPLSQALIPPELASAFKISPEPAKTMTDVNRASENTFSSLRGSGPLQQLQILSSPETEHGSDPRKTRRIDPEMLEADSALPRNENCGGGDDSNNKSASKTLNKRPRLVWTPQLHKRFVDVVAHLGIKNAVPKTIMQLMNVEGLTRENVASHLQKYRLYLKRMQGLSNEGPTSSDHLFAFTPVPQSLQQSGGNGQNGNGHNSSGYNSNGHNSNGHAQMPMQMPMPMPMIYPPQMVPMPMMGISGHGHGHGFHHQYNMGAQQRDWSGNKFGSYHHVAPSDE

SEQ ID NO: 12 - protein, 70 aa, Nicotiana tabacum
MGEKVKLSEYDSSGGNNDDRVLWEVGLPGADDLTPLTLQLIPAELASAFRISPESSKSMTDVIALLRILF

SEQ ID NO: 13 - protein, 185 aa, Lycopersicon esculentum
MGEEVSLTDYESSGGNDDRLLWEIGLPDVDDLTPLNMQLIPSELAAAFRISPELSKTMTDVNRASQNTFSSLQRWHSQDMASMNNSNFKTFSYERSREETVTERDETDLIREGSDSRKLRRVESGGTEEADSSLCNENFADDSSAKTLKRPRLVWTPQLHKRFIEVVAHLGIKGAVPKTIMQLMN

SEQ ID NO: 14 - protein, 314 aa, Solanum tuberosum
MGEEVNLTDYEYSGGNDDRLLWEVGLPDVDDLTPLTLQLIPSELAAAFRISPELSKTMTDVNRASQNTFSSLQRWHSQDMASMNNSYFKPFSYERTREETVTERDETDLIREGSDPRKLRRVESGGTEEADSSLCNENFADDSSAKTPKRPRLVWTPQLHKRFIEVVAHLGIKGAVPKTIMQLMNVEGLTRENVASHLQKYRLYTKRMQPNEGPSSSDHLFTSTPAAESMRESSEGGHLRNTNGHMAAPTLMPYQPQMVPMPMMGMPNGGHVVMPVGYGGGPPLGFHHHYNMVQQRDWSGNNFGYYHPVASNDK

SEQ ID NO: 15 - protein, 121 aa, Pinus taeda
  MISC_FEATURE (97)..(97): Xaa can be any naturally occurring amino acid
ERSIIDIDIDMKEEDWLAKWEEELPSPEELMPLTQNLITPDLAAAFKIHTSSASASAPTESGSVFPGSGQQQQRPGGEPPKRIELEEEESSVGGVTXNVGEEPARTLKRPRLVWTPQLHKR

SEQ ID NO: 16 - protein, 200 aa, Sorghum
MGEEAMDDYELHMVCYGGDDDGRVMEWESGLPGADELTPLSQPLVPPGLAAAFRIPPEPGRTLLDVHRASAATVSRLRRAPSSSSSGSGGSGSSSFAPFHPHPQAAAAAGHARGDEDADSSAAALGGGNAATTTTNTGTNSKRPRLVWTPQLHKRFVDVVAHLGIKNAVPKTIMQLMNVEGLTRENVASHLQKYRLYVKR